(19) United States
(12) Patent Application Publication
(10) Pub. No.: US 2024/0003908 A1
(43) Pub. Date: Jan. 4, 2024

(54) POLYOL BIOMARKERS FOR CONGENITAL DISORDERS OF GLYCOSYLATION

(71) Applicant: Mayo Foundation for Medical Education and Research, Rochester, MN (US)

(72) Inventors: Eva Morava-Kozicz, Rochester, MN (US); Tamas Kozicz, Rochester, MN (US); Devin Oglesbee, Rochester, MN (US); Kimiyo M. Raymond, Rochester, MN (US)

(21) Appl. No.: 18/036,031

(22) PCT Filed: Nov. 10, 2021
(86) PCT No.: PCT/US2021/058751

§ 371 Date: May 9, 2023

Related U.S. Application Data
(60) Provisional application No. 63/112,046, filed on Nov. 10, 2020.

(51) Int. Cl. G01N 33/68; A61P 3/00; A61K 31/426

(52) U.S. Cl. CPC G01N 33/6893; A61P 3/00; A61K 31/426; G01N 2440/38; G01N 2800/56

(57) ABSTRACT

Methods and materials for assessing the severity of congenital disorders of glycosylation (CDG), as well as methods and materials for treating CDG patients based on the assessment of CDG severity.

### CROSS-REFERENCE TO RELATED APPLICATIONS

This application claims benefit of priority from U.S. Provisional Application Ser. No. 63/112,046, filed on Nov. 10, 2020. The disclosure of the prior application is considered part of (and is incorporated by reference in) the disclosure of this application.

### STATEMENT AS TO FEDERALLY SPONSORED RESEARCH

This invention was made with government support under NS115198 awarded by the National Institutes of Health. The government has certain rights in the invention.

### TECHNICAL FIELD

This document relates to methods and materials for assessing the severity of congenital disorders of glycosylation (CDG), as well as methods and materials for treating CDG patients based on the assessment.

### BACKGROUND

CDG make up a group of inborn errors of metabolism that affect one of the most important post-translational modifications of proteins and lipids: glycosylation. A primary or secondary disturbance in any steps of this complex biochemical process can lead to a CDG. This orphan disorder group was first described in the 1980s (Jaeken et al., Pediatric Research 1980, 14:179), and more than 130 different types of CDG have since been identified. Type I CDG involve disrupted synthesis of the lipid linked oligosaccharide precursor (LLO) and its transfer to polypeptide chains. This type includes the subtypes phosphomannomutase-2-CDG (PMM2-CDG, also known as CDG-Ia), phosphomannose isomerase-CDG (MPI-CDG, also known as CDG-Ib), α-1,3-glucosyltransferase I-CDG (ALG6-CDG, also known as CDG-Ic), α-1,3-mannosyltransferase VI-CDG (ALG3-CDG, also known as CDG-Id), α-1,6-mannosyltransferase VIII-CDG (ALG12-CDG, also known as CDG-Ig), α-1,3-glucosyltransferase II-CDG (ALG8-CDG, also known as CDG-Ih), α-1,3-mannosyltransferase II-CDG (ALG2-CDG, also known as CDGIi), N-acetylglucosaminyltransferase I-CDG (DPAGT1-CDG, also known as CDG-Ij), β-1,4-mannosyltransferase I-CDG (ALG1-CDG, also known as CDG-Ik), α-1,2-mannosyltransferases VII/IX-CDG (ALG9-CDG, also known as CDG-Il), Flippase-CDG (RFT1-CDG, also known as CDG-1n), α-1,2-mannosyltransferases IV/V-CDG (ALG11-CDG, also known as CDG-Ip), oligosaccharyltransferase complex-CDG (DDOST-CDG, also known as CDG-Ir), Subunit TUSC3 of oligosaccharyltransferase complex-CDG (TUSC3-CDG), subunit MAGT1 of oligosaccharyltransferase complex-CDG (MAGT1-CDG), and steroid 5 alpha reductase type 3-CDG (SRD5A3-CDG). Type II CDG involve malfunctioning processing of the protein-bound oligosaccharide chain; this type includes subtypes β-1,2-N-acetylglucosaminylo-transferase II-CDG (MGAT2-CDG, also known as CDG-IIa), α-1,2-glucosidase I-CDG (MOGS-CDG, also known as CDG-IIb), phosphoglucomutase 1-CDG (PGM1-CDG, also known as CDG I T), signal sequence receptor subunit 4-CDG (SSR4-CDG, also known as CDG-Iy), solute carrier family 39 member 8-CDG (SLC39A8-CDG, also known as CDG IIn), and CDG type IIx. Other CDGs that are neither type I nor type II involve misregulation of GPI-anchors and lipid glycosylation, including phosphatidylinositol glycan anchor biosynthesis class N-CDG (PIGN-CDG) and phosphatidylinositol glycan anchor biosynthesis class S-CDG (PIGS-CDG).

The most frequently diagnosed CDG is PMM2-CDG, which has an incidence between 1:20,000 and 1:100,000 (Altassan et al., J Inherit Metab Dis. 2019, 42(1):5-28). Other types of CDG that are somewhat frequently diagnosed include MPI-CDG and ALG6-CDG. The clinical presentation in CDG ranges from developmental delay and neurologic symptoms to a severe multi-system disease that causes skeletal, cardiac, and endocrine abnormalities, coagulopathy, and failure to thrive. CDG patients commonly suffer from early, progressive peripheral neuropathy (Witters et al., Genetics Med. 2019, 21(5):1181-1188; and Altassan et al., supra). In most cases, losing the ability to walk as an adult is due to peripheral neuropathy combined with persistent muscle weakness and ataxia. In the first decade of life, neuropathy can progress from the absence of deep tendon reflexes to severe lower leg atrophy and the inability to take steps.

### SUMMARY

As demonstrated herein, patients with PMM2-CDG or other types of CDG have a disorder of activated monosaccharide balance that can affect their sugar alcohol (e.g., sorbitol and mannitol) synthesis. For example, as demonstrated in the Examples herein, patients with PMM2-CDG have elevated levels of urinary polyols such as sorbitol and mannitol, and the elevated urine polyol levels can be correlated with disease severity.

This document provides methods and materials for determining whether a mammal has, or is likely to have, a severe CDG, based on the level of one or more polyols in a biological sample from the mammal. For example, this document provides methods that include measuring the level of sorbitol in a biological sample (e.g., a urine sample) from a mammal identified as having a CDG, and identifying the mammal as having, or being likely to have, a severe CDG when the measured level of sorbitol is higher than a control level of sorbitol (e.g., the level of sorbitol in the same type of biological sample from a mammal of the same species that does not have the CDG). This document also provides methods and materials for treating a mammal identified as having, or being likely to have, a severe CDG. For example, a mammal that is identified as having, or being likely to have, a severe CDG using a method described herein, can be treated with a higher dose of an aldose reductase inhibitor (ARI) than would administered to a mammal identified as not having, or not being likely to have, a severe CDG.

Having the ability to determine whether a CDG patient has, or is likely to have, more severe disease provides a unique and unrealized opportunity to treat those patients more aggressively (e.g., by administering a higher dose of a therapeutic agent such as an ARI than would otherwise be administered). For example, a CDG patient identified as having, or being likely to have, severe CDG can be treated with a higher dose of an ARI such as epalrestat than would be given to a CDG patient who was determined not to have, or not likely to have, severe CDG.

In general, one aspect of this document features a method for identifying a mammal as having, or being likely to have, a severe CDG. The method can include, or consist essentially of, (a) measuring a level of one or more polyols in a biological sample from the mammal, and (b) determining that a measured level of at least one of the one or more polyols is elevated relative to a level of the one or more polyols in a biological sample from a control mammal that does not have the CDG, thereby identifying the mammal has having the severe CDG. The mammal can be a human. The CDG can be a Type II CDG. The CDG can be PMM2-CDG. The one or more polyols can include one or more of mannitol, sorbitol, maltitol, xylitol, and erythritol. For example, the measured polyol can be sorbitol. The biological sample can be a urine sample or a blood sample. The elevated level of the one or more polyols can be increased by at least 10% as compared to the level of the one or more polyols in the biological sample from the control mammal.

In another aspect, this document features a method for treating a mammal identified as (a) having a CDG and (b) having a biological sample containing an elevated level of one or more polyols, relative to the level of the one or more polyols in a control biological sample from a mammal not having the CDG. The method can include, or consist essentially of, administering to the mammal a dose of an aldose reductase inhibitor (ARI) that is increased as compared to a dose of the ARI that would be administered to a mammal identified as having the CDG and having a biological sample that does not contain an elevated level of the one or more polyols, relative to the level of the one or more polyols in the biological sample from the control mammal. The mammal can be a human. The CDG can be a Type II CDG. The CDG can be PMM2-CDG. The one or more polyols can include one or more of mannitol, sorbitol maltitol, xylitol, and erythritol. For example, the measured polyol can be sorbitol. The biological sample can be a urine sample or a blood sample. The ARI can be epalrestat, AT007, alrestatin, benurestat, epalrestat, fidarestat, imirestat, lidorestat, minalrestat, ponalrestat, ranirestat, risarestat, sorbinil, tolrestat, zenarestat, or zopolrestat. The elevated level of the one or more polyols can be increased by at least 10% as compared to the level of the one or more polyols in the control biological sample. The increased dose can be from about 0.5 mg/kg/day to about 5 mg/kg/day. The increased dose can be at least about 50% greater than a dose of the ARI previously administered to the mammal.

In another aspect, this document features a method for treating a mammal identified as having a CDG, where the method includes, or consists essentially of, (a) measuring a level of one or more polyols in a biological sample from the mammal, and determining that the measured level of at least one of the one or more polyols is elevated relative to a level of the one or more polyols in a control biological sample from a mammal not having the CDG, and (b) administering to the mammal a dose of an ARI that is increased as compared to a dose of the ARI that would be administered to a mammal identified as having the CDG and having a biological sample that does not contain an elevated level of the one or more polyols, relative to the level of said one or more polyols in said biological sample from said control mammal. The mammal can be a human. The CDG can be a Type II CDG. The CDG can be PMM2-CDG. The one or more polyols can include one or more of mannitol, sorbitol maltitol, xylitol, and erythritol. For example, the measured polyol can be sorbitol. The biological sample can be a urine sample or a blood sample. The ARI can be epalrestat, AT007, alrestatin, benurestat, epalrestat, fidarestat, imirestat, lidorestat, minalrestat, ponalrestat, ranirestat, risarestat, sorbinil, tolrestat, zenarestat, or zopolrestat. The elevated level of the one or more polyols can be increased by at least 10% as compared to the level of the one or more polyols in the control biological sample. The method increased dose can be from about 0.5 mg/kg/day to about 5 mg/kg/day. The increased dose can be at least about 50% greater than a dose of the ARI previously administered to the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B are graphs plotting the correlation between urine sorbitol (FIG. 1A) and mannitol (FIG. 1B) levels and neuropathy in patients with PMM2-CDG. Mild neuropathy included decreased deep tendon reflexes and no muscle atrophy; moderate neuropathy included decreased reflexes or areflexia with distal weakness and distal muscle atrophy, but mobility; and severe neuropathy included areflexia and full reliance on mobility aids, primarily due to the neuropathy.

FIGS. 2A-2D are a series of graphs plotting the correlation between urine sorbitol levels and neuropathy (FIG. 2A; p=0.0012, r=0.7), Nijmegen Patient CDG Rating Scale (NPCRS) severity score (FIG. 2B; p-0.0002, r=0.55), age (FIG. 2C), and gender (FIG. 2D) in 20 patients with PMM2-CDG. Male/Female is denoted by a value of either 1 or 2. Points with a y score of 1 are female, points with a y score of 2 are male.

FIG. 3 is a graph plotting urine sorbitol levels in a PMM2-CDG patient on epalrestat therapy. Prior to treatment, the patient's urine sorbitol level was increased, measuring 19.93 mmol/mol creatinine (control levels were <5; not shown). After 4 months of epalrestat treatment, the patient's sorbitol level decreased to 5.0 mmol/mol creatinine. At follow up (6 months; not shown), the patient's urine sorbitol level was 6.2 mmol/mol creatinine.

FIGS. 4A-4C are graphs plotting polyol, mannitol, and sorbitol levels in fibroblasts from PMM2-CDG patients as compared to healthy controls, as determined by gas chromatography-mass spectrometry (GC-MS). The polyol pool (FIG. 4A), mannitol levels (FIG. 4B) and sorbitol levels (FIG. 4C) were elevated in PMM2-CDG fibroblasts as compared to controls. Cells were cultured in normal glucose (GLC) containing medium (4.6 M).

FIGS. 5A and 5B are a pair of graphs plotting mannitol and sorbitol levels in fibroblasts from PMM2-CDG patients and healthy controls (determined by GC-MS) after treatment for 5 days with epalrestat. Both mannitol (FIG. 5A) and sorbitol (FIG. 5B) levels were decreased in fibroblasts treated with epalrestat.

FIGS. 6A-6E show that epalrestat treatment increased PMM enzyme activity and ICAM-1 protein abundance. FIG. 6A is a graph plotting PMM enzyme activity in PMM2-CDG patient fibroblasts (n=11; P1-P6, P8, P10, P17, P19, P24) treated with vehicle or 10 μM epalrestat treatment for 24 hours. FIG. 6B is a graph plotting ICAM-1 protein abundance in immunoblots with PMM2-CDG patient fibroblasts, based on band intensity (p=0.02; n=10; P1-P6, P8, P17, P19, P24) treated with vehicle or epalrestat. FIG. 6C is a graph plotting LAMP-2 protein abundance in immunoblots with PMM2-CDG patient fibroblasts (n=10; P1-P6, P8, P17, P19, P24) treated with vehicle or epalrestat. FIG. 6D contains images of immunoblots showing ICAM-1 protein abundance in untreated and epalrestat-treated patient fibroblasts (n=10; P1-P6, P8, P17, P19, P24), with beta-actin as a loading control. FIG. 6E contains images of immunoblots showing LAMP-2 protein abundance in untreated and epalrestat-treated patient fibroblasts (n=10; P1-P6, P8, P17, P19, P24), with beta-actin as a loading control.

FIGS. 7A-7D show proteomic changes in PMM-deficient patient-derived fibroblasts and effects of epalrestat treatment. FIG. 7A is a waterfall plot of global proteomics of PMM2-CDG fibroblasts and controls. The Y-axis represents log2 fold changes (PMM2-CDG/controls) and the X-axis represents the number of proteins identified. Each individual circle represents a protein. Names are provided for some of the highly changing representative proteins. FIG. 7B is a volcano plot for the same comparison as shown in FIG. 7A. The X-axis represents the log2 fold change (PMM2-CDG/controls) and the Y-axis represents the negative logarithm of the p value of a t-test for significance. The horizontal dashed line marks the cutoff for significance (<0.05), and the vertical dashed lines highlight the proteins having at least a 30% change in either direction (1.3-fold enhancement or reduction). Names are provided for some of the most significantly changing proteins. FIG. 7C is a waterfall plot for a paired comparison of PMM-deficient fibroblasts treated with epalrestat or vehicle. The Y-axis represents the log2 fold changes (epalrestat-treated/untreated) and the X-axis represents the number of proteins identified. Each identified protein is depicted with a circle, and names are provided for some of the highly changing proteins. FIG. 7D is a volcano plot for the treated/untreated comparison of PMM-deficient fibroblasts. The X-axis depicts the log2 fold change (treated/untreated) and the Y-axis is the negative logarithm of the p value of a t-test for significance. The horizontal dashed line marks the cutoff for significance (paired t-test, <0.05) and the vertical dashed lines highlight the proteins having at least a 30% change in either direction (1.3-fold enhancement or reduction). Names are provided for some of the proteins showing relative higher abundance upon epalrestat treatment.

FIGS. 8A-8D show glycoproteome alterations in PMM-deficient fibroblasts and remodeling after epalrestat treatment. FIG. 8A is a waterfall plot of global glycoproteomics for PMM-deficient fibroblasts and controls. The Y-axis represents log2 fold changes (PMM2-CDG/controls) and the X-axis represents the number of unique glycopeptides identified. Each individual circle represents a unique glycopeptide (a unique combination of peptide and glycan structure). Glycoprotein names are provided for some of the highly changing representative glycopeptides, with glycosylation sites (N with the corresponding amino acid number) and plausible glycan structures shown. The oval in the lower portion (the negative Y-axis) of the waterfall plot depicts unique glycopeptides, having the plausible glycan structures shown in the box, that were reduced in PMM-deficient fibroblasts. FIG. 8B is a volcano plot for the comparison of PMM2-CDG to controls. The X-axis represents the log2 fold change (PMM2-CDG/controls) and the Y-axis is the negative logarithm of the p value of a t-test for significance. The horizontal dashed line marks the cutoff for significance (<0.05) and the vertical dashed lines highlight the glycoproteins having at least a 30% change in either direction (1.3-fold enhancement or reduction). Names are provided for some of the highly changing glycopeptides, with glycosylation sites and plausible glycan structures shown. FIG. 8C is a waterfall plot depicting comparative glycoproteomics for PMM-deficient fibroblasts treated with epalrestat or vehicle. The Y-axis represents the log2 fold change (epalrestat-treated/untreated) and the X-axis represents the number of unique glycopeptides identified and quantified. Each unique glycopeptide is depicted with a black circle, names are provided for some of the altered glycopeptides, with glycosylation sites and plausible glycan structures shown. FIG. 8D is a volcano plot showing the comparison of treated/untreated glycoproteomics of PMM-deficient fibroblasts. The X-axis represents the log2 fold change (treated/untreated) and the Y-axis represents the negative logarithm of the p value of a t-test for significance. The horizontal dashed line marks the cutoff for significance (paired t-test, <0.05) and the vertical dashed lines highlight the glycopeptides having at least a 30% change in either direction (1.3-fold enhancement or reduction). Using this cutoff, names and plausible glucan structures for some of the glycopeptides showing enhanced levels upon epalrestat treatment are provided. None of the unique glycopeptides was found to be reduced.

FIG. 9 is a waterfall plot showing paired comparisons at the protein level for PMM-deficient fibroblasts treated with epalrestat or vehicle. The Y-axis represents the log2 fold changes (epalrestat-treated/untreated), and the X-axis represents the number of proteins identified. Each identified protein is depicted with a black circle, names for some of the highly changing proteins are provided.

FIG. 10 is a graph plotting the log2 fold changes for the glycopeptides that exhibited the highest increase in abundance (%) after epalrestat treatment. Epalrestat treated (solid line) and untreated (dashed line) PMM2-CDG patient-derived fibroblasts were compared to control fibroblasts. The ten glycopeptides that showed the greatest percent increase in their relative abundance are shown. Each data point represents one glycopeptide. The corresponding protein names, glycosylation sites, and plausible glycan structures are provided below the graph.

FIGS. 11A-11F are graphs showing the association of urine sorbitol and mannitol concentrations (normalized to urine creatinine concentration) with peripheral neuropathy, liver pathology, and CDG phenotype. Significant variations in urine sorbitol concentrations were associated with both peripheral neuropathy score (FIG. 11A) and liver pathology score (FIG. 11B), with elevated urine sorbitol detected in CDG patients displaying both moderate neuropathy and mild liver pathology. Significant variations in urine mannitol concentrations also were associated with both peripheral neuropathy score (FIG. 11C) and liver pathology score (FIG. 11D), with elevated urine mannitol detected in CDG patients displaying both moderate neuropathy and mild liver pathology. Significance was assessed by Kruskal-Wallis test followed by Dunn's multiple comparisons test (FIGS. 11A and 11C) and Mann Whitney test (FIGS. 11B and 11D). Data are expressed as mean±SD. p<0.05(*), p<0.01(**), p<0.001(**). FIG. 11E is a series of plots showing that urine sorbitol levels were positively correlated with severe CDG phenotype (r=0.5, p<0.02), and that there was no correlation with the mild or moderate category. Comparison was done following normalization of sorbitol to urine creatinine concentration. FIG. 11F is a series of plots demonstrating that urine mannitol levels did not correlate with mild, moderate, or severe categories based on NPCRS scores. Comparison was done following normalization of mannitol to urine creatinine concentration.

FIGS. 12A-12E show that epalrestat treatment had a positive effect on glycosylation defect, growth, and normalization of elevated sorbitol and mannitol levels in a PMM2-CDG pediatric patient. FIG. 12A is a graph plotting the BMI of the patient over time, before and after epalrestat treatment. FIG. 12B is a graph plotting a pharmacokinetics (PK) profile for epalrestat, showing rapid elimination (t½˜1-2 hours). FIG. 12C is a graph plotting urine sorbitol levels before starting epalrestat therapy (sorbitol=19.93 mmol/mol creatinine), and after 3 months (5.78 mmol/mol creatinine) and 6 months of therapy (6.20 mmol/mol creatinine) as compared to controls (<5 mmol/mol creatinine). FIG. 12D is a graph plotting urine mannitol levels before starting epalrestat therapy (mannitol=648.6 mmol/mol creatinine) and after 3 months (37.32 mmol/mol creatinine) and 6 months of therapy (25.09 mmol/mol creatinine) as compared to controls (<20 mmol/mol creatinine). FIG. 12E is a graph plotting weight and blood transferrin glycoform ratio analysis over time. Weight increased during epalrestat therapy, while blood transferrin glycoform ratio decreased 12 months prior to therapy and during 12 months of epalrestat therapy. Normal levels for mono-oligo/di-oligo controls: ratio≤0.06; normal levels for A-oligo/di-oligo controls: ratio≤0.01.

FIG. 13 is a graph plotting delta fold changes for 412 glycopeptides that showed increased abundance after epalrestat treatment (calculated by subtracting the fold changes of non-responders from the fold changes of responders). The graph shows the difference in glycosylation increase between responders and non-responders, where each circle denotes one individual glycopeptide and the X-axis depicts the number of glycopeptides.

### DETAILED DESCRIPTION

CDG are a group of inborn errors of metabolism that affect glycosylation, an important post-translational modification of proteins and lipids. A disturbance in any steps of this complex biochemical process can lead to a CDG. The most frequently diagnosed CDG is PMM2-CDG. No cure is currently available for PMM2-CDG, but it has been shown that epalrestat can increase PMM2 activity in PMM2-CDG patient fibroblasts in vitro (Iyer et al., bioRxiv 2019, 626697).

Epalrestat is a carboxylic acid derivative that is a noncompetitive and reversible aldose reductase inhibitor (ARI), and has been used for the treatment of diabetic neuropathy. Aldose reductase (ALR2) is a key enzyme that is involved in the polyol (sorbitol) pathway, activating the conversion of glucose into sorbitol and subsequently to fructose. ALR2 plays a crucial role in the development of diabetic peripheral neuropathy, hyperglycemia, and alcoholic liver disease (ALD) by suppressing inflammatory cytokines and lipid metabolism (Srivastava et al., Chem Biol Interact. 2011, 191(1-3):330-338), and by acting as an obligatory mediator of oxidative stress. Epalrestat is easily absorbed into neural tissue, and can inhibit ALR2 with minimal side effects. The drug has been used in diabetic neuropathic pain management (Haneda et al., Diabetol Int 2018, 9:1-45), and has demonstrated efficacy in decreasing sorbitol levels in diabetic patients to delay neuropathy progression without reported complications (Hotta et al., Diabet Med 2012, 29(12): 1529-1533).

As described in the Examples herein, patients with PMM2-CDG had elevated levels of urinary polyols, and the increase in polyol levels was correlated with increased severity of disease and/or disease-associated neuropathy. Polyols are sugar alcohols, and have been identified in blood, urine, and cerebrospinal fluid. Examples of polyols include, without limitation, sorbitol, mannitol, maltitol, xylitol, and erythritol.

This document provides methods and materials for identifying and/or treating CDG patients who have, or have an increased likelihood of developing, more severe disease. As used herein, the term “more severe disease” refers to more severe multisystem disease, including peripheral neuropathy, associated with a CDG. The methods and materials provided herein can be used, for example, to identify a mammal (e.g., a human) with a CDG as being at increased risk of having or developing more severe multisystem disease, including peripheral neuropathy.

Any appropriate mammal having a CDG can be identified as being at increased risk of more severe disease, using the materials and methods provided herein. For example, humans and other primates such as monkeys having CDG can be identified as having an increased likelihood of having or developing more severe disease. In some cases, dogs, cats, horses, cows, pigs, sheep, mice, or rats having CDG can be identified as having an increased likelihood of having, or progressing to, more severe disease.

As described herein, a mammal (e.g., a human) with a CDG (e.g., PMM2-CDG) can be identified as having an increased likelihood of having or developing more severe disease by detecting an increased level of one or more polyols in a biological sample from the mammal. Examples of polyols that can be evaluated and used to classify a mammal (e.g., a human) as having (or not having) an increased likelihood of having or developing more severe disease include, without limitation, sorbitol, mannitol, and galactitol. A polyol level can be measured in any appropriate biological sample. Suitable biological samples include, without limitation, urine, blood, serum, cerebrospinal fluid, and tissue or cell samples (e.g., fibroblasts).

Biological samples can be obtained via any appropriate method (e.g., biopsy, brushing, swabbing, scraping, or fluid collection). Any appropriate method can be used to determine if a mammal (e.g., a human) has an elevated level of one or more of the markers (e.g., sorbitol and/or mannitol) listed herein. The term “elevated level” as used herein with respect to the level of a polyol marker refers to a level of the marker in a sample that is greater (e.g., at least 5, 10, 25, 35, 45, 50, 55, 65, 75, 80, 90, 100, 200, 300, 400, 500, 600, or more than 600 percent greater) than the median level of that marker in a corresponding biological sample from an unaffected mammal (e.g., a “normal” or “healthy” mammal) identified as not having the CDG, where the control mammal is of the same species as the mammal being tested.

Appropriate methods for identifying biological samples as having an elevated level of one or more polyol markers described herein include, without limitation, GC/MS, LC/MS, matrix-assisted laser desorption/ionization-time of flight (MALDI-TOF) mass spectrometry, and quadruple time of flight (QTOF) mass spectrometry. In some cases, for example, a urine sample from a mammal (e.g., a human) identified has having a CDG can be assessed by GC/MS to measure the level of sorbitol or mannitol in the sample, and the measured level can be compared with the level measured in a urine sample from a control mammal of the same species that does not have the CDG. In some cases, a blood sample from a mammal (e.g., a human) identified has having a CDG can be assessed by GC/MS to measure the level of sorbitol and/or mannitol in the sample, and the measured level can be compared with the level measured in a blood sample from a control mammal of the same species that does not have the CDG.

Once a mammal (e.g., a human) is identified as having a biological sample with an elevated level of one or more polyol markers, the mammal can be classified as being likely to have severe CDG, or as having an increased risk of developing severe CDG (e.g., more severe multisystem disease, including peripheral neuropathy). For example, a human identified as having a sample (e.g., a urine sample) with increased levels of one or more (e.g., one, two, three, four, five, or more than five) polyols can be classified as having severe disease, or as being at increased risk of developing severe disease. In some cases, a mammal identified as having a biological sample with an increased overall level of polyols (e.g., an increased polyol pool) can be classified as having severe disease, or as being at increased risk of developing severe disease. In some cases, a mammal (e.g., a human) identified as having a biological sample that does not exhibit an increased level of one or more (e.g., one, two, three, four, five, or more than five) polyol markers can be classified as not having, or not being at increased risk of developing, severe CDG.

This document also provides methods for treating mammals identified as having, or being at increased risk for developing, a more severe CDG. In addition, this document provides methods for modifying the treatment of mammals that are undergoing treatment for a CDG and are identified as having, or being at increased risk for developing, severe disease. As noted above, mammals with CDG can be treated with an ARI. Examples of ARIs that can be administered to mammals with CDG include, without limitation, epalrestat, AT007, alrestatin, benurestat, epalrestat, fidarestat, imirestat, lidorestat, minalrestat, ponalrestat, ranirestat, risarestat, sorbinil, tolrestat, zenarestat, and zopolrestat.

In some cases, a mammal identified as having a CDG and having, or being at increased risk of developing, severe disease, can be treated with an ARI at a dose higher than the dose that would be administered to a mammal newly diagnosed with a CDG. In some cases, a mammal identified as having a CDG and having, or being at increased risk of developing, severe disease, can be treated with an ARI at a dose higher than the dose that would be administered to a mammal that does not have a severe CDG. In some cases, the treatment course of a mammal that is already undergoing treatment with an ARI can be adjusted such that the dosage of the ARI and/or the frequency of administration is increased when the mammal is identified as having a biological sample with an elevated level of one or more polyols (e.g., sorbitol and/or mannitol). Having the ability to use a more aggressive treatment (e.g., an increased dosage, increased frequency of administration, or both) to achieve better disease control can allow clinicians and patients to proceed with treatment options that mitigate the risk for severe CDG. In addition, mammals with increased risk for severe CDG can undergo more regular surveillance (e.g., examination and/or sample testing on a more frequent basis) to detect early changes in CDG disease course.

In some cases, an effective amount of an ARI (e.g., epalrestat, AT007, alrestatin, benurestat, epalrestat, fidarestat, imirestat, lidorestat, minalrestat, ponalrestat, ranirestat, risarestat, sorbinil, tolrestat, zenarestat, and zopolrestat) can be administered to a mammal once or more than once over a period of time ranging from days to months. Effective doses can vary depending on the severity of the CDG, the route of administration, the age and general health condition of the subject, excipient usage, the possibility of co-usage with other therapeutic treatments, and the judgment of the treating physician. An effective amount of a composition containing an ARI (e.g., epalrestat, AT007, alrestatin, benurestat, epalrestat, fidarestat, imirestat, lidorestat, minalrestat, ponalrestat, ranirestat, risarestat, sorbinil, tolrestat, zenarestat, and zopolrestat) can be any amount that reduces the likelihood that the CDG will progress to severe disease, or any amount that reduces disease symptoms, without producing significant toxicity to the mammal. The current trialed dose of epalrestat in PMM2 children is about 0.8 mg/kg/day, while the current dose for adults is about 3 mg/kg/day, but an effective amount of an ARI (e.g., epalrestat, AT007, alrestatin, benurestat, epalrestat, fidarestat, imirestat, lidorestat, minalrestat, ponalrestat, ranirestat, risarestat, sorbinil, tolrestat, zenarestat, and zopolrestat) can be from about 0.1 mg/kg/day to about 10 mg/kg/day (e.g., from about 0.1 to about 0.3 mg/kg/day, from about 0.3 to about 0.5 mg/kg/day, from about 0.5 to about 0.8 mg/kg/day, from about 0.8 to about 1 mg/kg/day, from about 1 to about 1.5 mg/kg/day, from about 1.5 to about 2 mg/kg/day, from about 2 to about 3 mg/kg/day, from about 3 to about 4 mg/kg/day, from about 4 to about 5 mg/kg/day, from about 5 to about 7 mg/kg/day, or from about 7 to about 10 mg/kg/day).

As described herein, however, a mammal identified as having, or being at increased risk of developing, severe CDG can be treated with an increased dose of an AIR. A “higher” or “increased” dose of an ARI can be, for example, a dose that is increased by at least 25% (e.g., at least 50%, at least 100%, at least 150%, 200%, at least 300%, at least 400%, or more than 400%) as compared to the dose currently being administered to a mammal, or as compared to a standard or trialed dose that would be administered to the mammal. Again, the current trialed dose of epalrestat in PMM2 children is about 0.8 mg/kg/day, while the current maximum standard dose for adults is about 3 mg/kg/day. In some cases, the dose in a child identified as being likely to have or develop more severe disease can be increased to at least about 1 mg/kg/day (e.g., at least 1.5 mg/kg/day, at least 2 mg/kg/day, at least 2.5 mg/kg/day, at least 3 mg/kg/day, from about 1 to about 1.5 mg/kg/day, from about 1.5 to about 2 mg/kg/day, from about 2 to about 2.5 mg/kg/day, or from about 2.5 to about 3 mg/kg/day), while the dose in an adult identified as being likely to have or develop more severe disease can be increased to at least about 4 mg/kg/day (e.g., at least about 5 mg/kg/day, at least about 7.5 mg/kg/day, at least about 10 mg/kg/day, at least about 12.5 mg/kg/day, or at least about 15 mg/kg/day).

In some cases, if a mammal fails to respond to a particular dosage of an agent (e.g., an ARI administered at a higher dosage than a trialed or standard dosage previously administered to the mammal), then the amount of the administered agent can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition may require an increase or decrease in the actual effective amount administered.

The frequency of administration of an agent (e.g., an ARI) to a mammal having a CDG can be any frequency that reduces the symptoms of the CDG, or that reduces the likelihood that the CDG will progress to a severe disease, without producing significant toxicity to the mammal. For example, the frequency of administration can be from about once a day to about once a month (e.g., from about once a week to about once every other week). The frequency of administration can remain constant or can be variable during the duration of treatment. A course of treatment with a composition containing one or more agents (e.g., an ARI such as epalrestat) can include rest periods. For example, a composition containing an ARI can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition may require an increase or decrease in administration frequency.

An effective duration for administering a composition containing one or more agents (e.g., an ARI) to a mammal having a CDG can be any duration that alleviates one or more symptoms of the CDG, or that reduces the likelihood that the CDG will progress to a severe disease without producing significant toxicity to the mammal. In some cases, the effective duration can vary from months to years. Since CDG are congenital disorders for which there is currently no curative treatment, in some cases, the effective duration of treatment can be life-long. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and/or the severity of one or more symptoms related to the condition being treated (e.g., PMM2-CDG) can be monitored. Any appropriate method can be used to determine whether or not a mammal's likelihood of developing severe disease has been delayed or reduced. For example, one or more biological samples (e.g., urine samples) can be assessed following the onset of administration of an agent (or onset of treatment modification to increase the dosage, for example) to determine if the treatment reduced the level of one or more of the polyol markers in the samples, as compared to previously measured levels. In some cases, changes in protein glycosylation (e.g., total protein N-glycosylation) in a mammal having a CDG and treated with an ARI can be monitored to assess the effectiveness of the treatment. For example, an increase in protein glycosylation levels after treatment with an ARI can serve as an indication that the treatment was effective to delay or reduce the mammal's likelihood of developing severe disease. In some cases, a combination of factors (e.g., urine polyol levels, protein glycosylation levels, and/or disease symptoms) can be monitored to determine whether or not a mammal's likelihood of developing severe disease has been delayed or reduced. Any appropriate methods can be used to assess polyol and/or protein glycosylation levels, including, for example, the methods described in the Examples herein.

The invention will be further described in the following examples, which do not limit the scope of the invention described in the claims.

### EXAMPLES

### Example 1—Correlation of Sorbitol and Mannitol Levels with PMM2-CDG Severity

### Methods

Studies were conducted to investigate sorbitol and mannitol levels in the urine of 20 patients with PMM2-CDG. In addition, sorbitol excretion in urine was investigated in a single PMM2-CDG patient on epalrestat therapy, sorbitol and mannitol levels in PMM2-CDG fibroblasts were measured and compared to healthy controls, and fibroblasts were treated with epalrestat to evaluate its potential for treating elevated sorbitol and mannitol levels in PMM2-CDG.

Metabolomics measurements: Fibroblasts derived from patients carrying selected heterozygous mutations in PMM2 (R141H/F119L, R141H/E139K, R141H/N216I, and R141H/F183S) and healthy controls were cultured according to a protocol described elsewhere (Radenkovic et al., Am J Hum Genet. 2019, 104(5):835-846). Cells were grown in specialized medium and treated with 10 μM epalrestat (Iyer et al., supra) for 5 days with two changes of medium. Non-treated fibroblasts from both groups were used as negative controls. Global metabolomic changes were probed by LC/MS.

Polyols also were analyzed by GC due to the inability of LC/MS to distinguish between the same sized metabolites (M1p<M6P, GLC1P, GLC6P, sorbitol/mannitol/galactitol, etc.) (Radenkovic et al., supra).

Clinical studies: Epalrestat was used as an N of 1 study for a single pediatric patient with PMM2-CDG. An open label, single patient compassionate use study designed to assess the safety and tolerability of oral epalrestat therapy in a child with PMM2-CDG. The patient received 0.8 mg/kg of epalrestat three times per day. Concomitant medications, vital signs, blood draw for serum chemistry and hematology, and blood levels of epalrestat were collected at each study visit during a 6 month period to evaluate the safety and tolerability of oral epalrestat therapy in a child with PMM2-CDG. The Nijmegen Pediatric CDG Rating Scale (NPCRS) assessment was administered at the start of the study and at 6 months. Urine polyols were collected at the start, at 4 months, and at 6 months of therapy, and were measured as described elsewhere (Jansen et al., Clin Chim Acta 1986, 157:277-294). Briefly, 200 μl urine specimens were spiked with a mixture of labeled internal standards, allowed to equilibrate, and evaporated. The dry residue was derivatized to form trimethylsilyl (TMS) esters then extracted with hexane. Specimens were analyzed by GC/MS, selected ion monitoring using ammonia chemical ionization and a stable isotope dilution method. In general, the following protocol was used:
Creatinine (CR) values (mg/dL) were determined for all samples.
Multiplication factors were calculated for each patient sample.
100 μL of Polyol QC Mix for the QC Standard and 100 μL water were pipetted into a labeled 16×100 screw-cap glass tube.
200 μL each of low control and high control were pipetted into corresponding labeled 16×100 mm screw-cap glass tubes.
200 μL of each patient sample was pipetted into a corresponding labeled 16×100 mm screw cap glass tube. For patient samples requiring dilution, the multiplier was adjusted.
100 μL of Polyol Internal Standard was pipetted into each of the control and patient tubes, and tubes were vortexed to mix.
The liquid was evaporated by placing the tubes in a 60° C. evaporator. Each sample was evaporated just to dryness under a gentle nitrogen flow, with careful monitoring.
100 μL of Tri-Sil-TBT was pipetted into each tube. Tubes were capped, vortexed, and placed in an 80° C. heating block for 30 minutes.
The tubes were removed from the heating block and allowed to cool to room temperature.
1 mL of 0.1 M HCl was slowly pipetted into each tube with caution, as the derivatizing reagent may react strongly upon the addition of 0.1 M HCl.
1 mL of hexane was added to each tube. Tubes were mixed by vortexing for about 10 seconds, and then centrifuged at 2000 RPM for 1 minute.
The hexane (upper) layer was removed from each tube (with care to avoid contact with the lower aqueous phase) and placed into a corresponding labeled 16×100 tube.
0.5 g Na₂SO₄ was added to each of the extraction tubes.
The extraction tubes were vortexed for about 10 seconds and then let stand at room temperature for 2 minutes to allow the Na₂SO₄ to remove any residual water. Tubes were then centrifuged for 1 minute at 2000 RPM.
200 μL of hexane was pipetted from each tube into a labeled glass GC vial for GC/MS analysis.

Urine sorbitol and mannitol levels also were quantified using similar processing of samples and GC/MS settings, monitoring ions specific for sorbitol and mannitol for peak area ratio calculations.

### Results

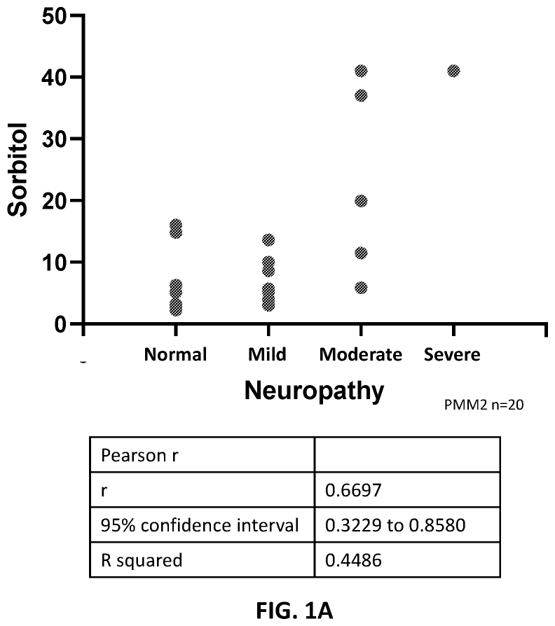

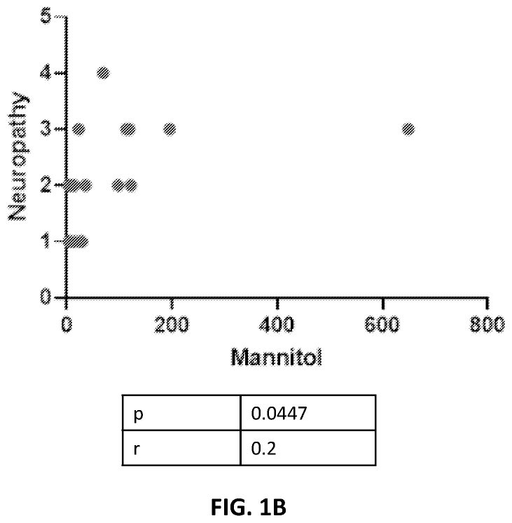

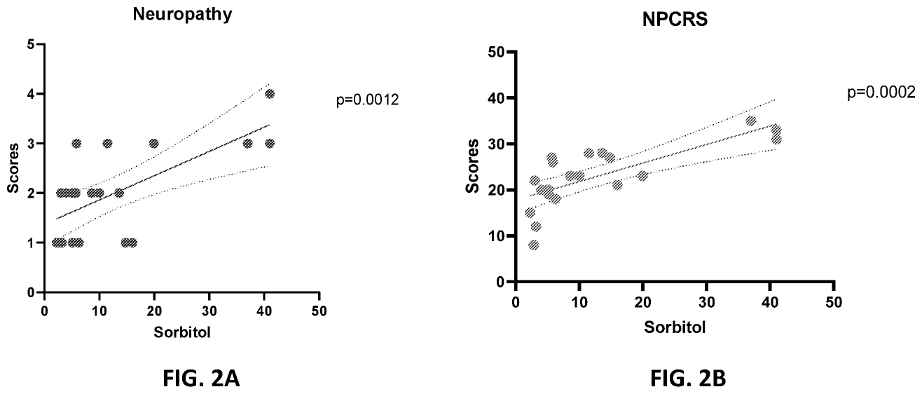

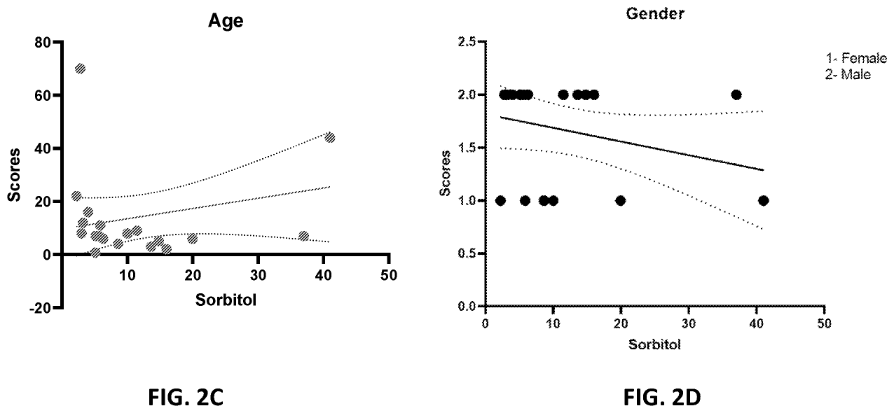

The studies described above demonstrated that sorbitol levels were elevated in urine from patients with PMM2-CDG, and were correlated with disease severity and neuropathy. Sorbitol levels ranged from 2.24 to 41 mmol/mol creatinine (controls <5 mmol/mol creatinine). Overall disease severity was assessed by the Nijmegen Patient CDG Rating Scale (NPCRS), a validated clinical scale to measure overall disease severity in CDG. The severity of neuropathy in patients was scored between 1 (no neuropathy) and 4 (severe neuropathy), adapted from the NPCRS. Urine sorbitol levels showed a correlation with both the degree of severity of the neuropathy (FIGS. 1A and 2A) and disease severity according to NPCRS (FIG. 2B). There was no correlation between urine sorbitol levels and age or gender (FIGS. 2C and 2D, respectively).

Urine mannitol levels also were elevated in patients with PMM2-CDG, and were correlated with severity of neuropathy. Mannitol levels ranged from 3.64 to 648.6 mmol/mol creatinine (controls <10 mmol/mol creatinine). Overall disease severity was assessed by the NPCRS, and the severity of neuropathy was scored between 1 (no neuropathy) and 4 (severe neuropathy). Urine mannitol levels showed no correlation with disease severity according to NPCRS, but did show a correlation with the degree of severity of the neuropathy (FIG. 1B).

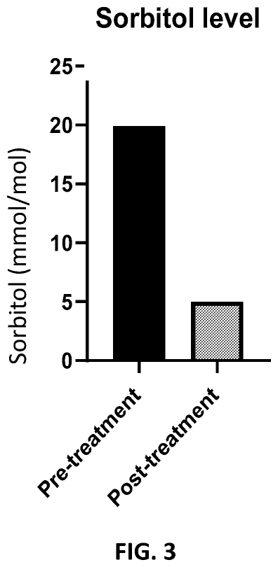

Urine sorbitol levels in a 7 year-old patient with PMM2-CDG were elevated prior to treatment, but improved to close to normal levels after 4 to 6 months of oral Epalrestat therapy (FIG. 3). Prior to treatment, the patient's urine sorbitol level was 19.93 mmol/mol creatinine, while control levels were <5 mmol/mol creatinine. After 4 months of epalrestat treatment, the patient's sorbitol level decreased to 5.0 mmol/mol creatinine (FIG. 3). At a 6 month follow up, the patient's urine sorbitol level was 6.2 mmol/mol creatinine. The patient's urinary mannitol levels also were decreased after epalrestat treatment; the pretreatment level was 648.6 mmol/mol creatinine, and the level at 4 months was decreased to 37.32 mmol/mol creatinine after epalrestat treatment. The control level was less than 10 mmol/mol creatinine.

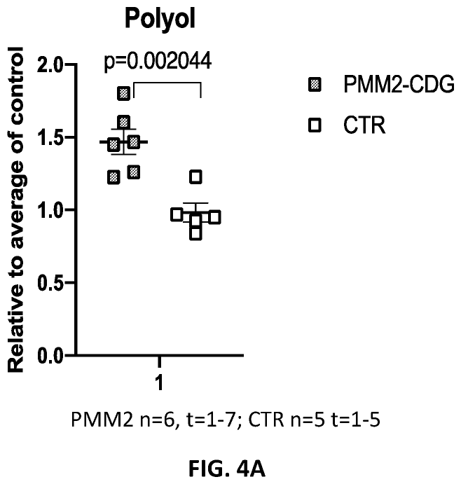

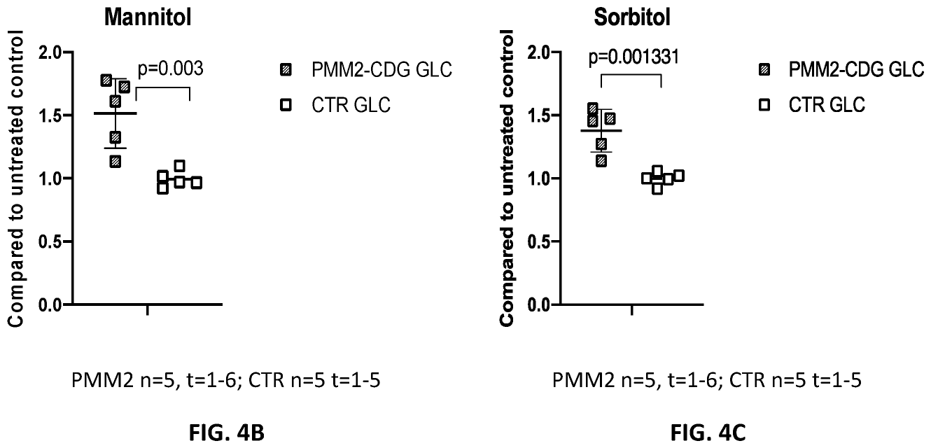

The studies utilizing fibroblasts demonstrated that the overall polyol pool, as well as sorbitol and mannitol levels specifically, were increased in vitro in fibroblasts from PMM2-CDG patients as compared to fibroblasts from healthy controls. Polyols were measured by LC/MS in fibroblasts from 6 PMM2-CDG patients and 7 healthy controls. The polyol concentration compared to controls was significantly elevated (mean ratio 1.48, p=0.0002; FIG. 4A). Sorbitol concentrations were measured by GC/MS in fibroblasts from 5 PMM2-CDG patients and 6 healthy controls. The sorbitol concentration compared to controls was significantly elevated (mean ratio 1.4, p=0.00013; FIG. 4B). Mannitol concentrations were measured by GC/MS in fibroblasts from 5 PMM2-CDG patients and 6 healthy controls. The mannitol concentration compared to controls was significantly elevated (mean ratio 1.5, p=0.0003; FIG. 4C).

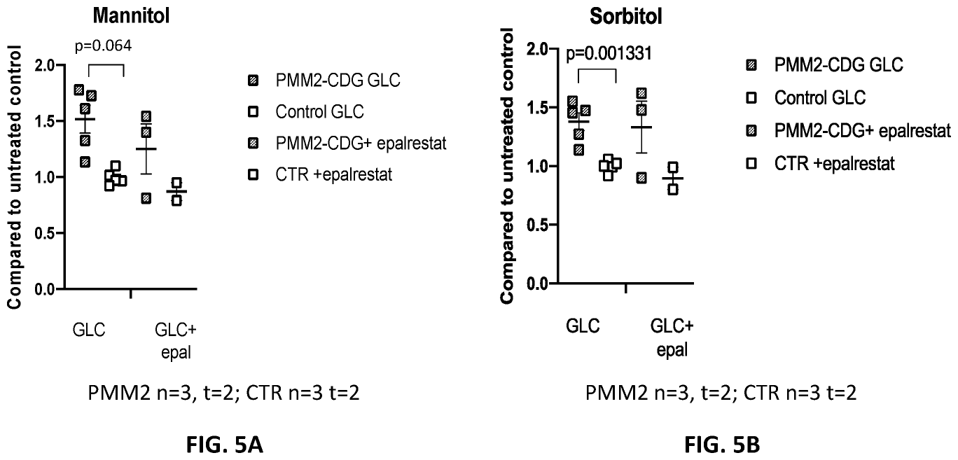

Treatment of patient fibroblasts with epalrestat for 5 days led to decreased levels of sorbitol and a trend toward decreased levels of mannitol, such that the levels of sorbitol in patient fibroblasts were no longer significantly greater than the levels in fibroblasts from healthy controls. Polyols were measured by GC/MS in fibroblasts from 5 PMM2-CDG patients and 5 healthy controls. The mannitol concentration after treatment, as compared to the concentration prior to treatment, trended toward improved mannitol levels (mean ratio 1.2 to 1.5, p=0.064; FIG. 5A). The sorbitol concentration after treatment, as compared to the concentration prior to treatment, was significantly improved (mean ratio 1.25 to 1.4, p=0.00013; FIG. 5B).

### Example 2—Elevated Polyols as Markers in Other CDG

Urine specimens were collected from patients known to have a CDG. The samples were spiked with a mixture of labeled internal standards, allowed to equilibrate, and evaporated. The dry residue was derivatized to form trimethylsilyl esters then extracted with hexane. Specimens were analyzed by GC/MS, with selected ion monitoring using ammonia chemical ionization and a stable isotope dilution method (Jansen et al. Clin Chim Acta 1986, 157:277-294; and Kaur et al., Eur J Med Genet 2019, 62(8):103708). Sorbitol levels were considered elevated when they were above 10 mmol/mol creatinine, and mannitol levels were considered elevated when they were above 15 mmol/mol creatinine.

These studies demonstrated that elevated polyols were not exclusively a marker of PMM2-CDG. Patients with MAN1B1-CDG, ALG6-CDG, SLC39A8-CDG, MOGS-CDG, CDG type IIx, NANS-CDG, PIGS-CDG, and ALG8-CDG also demonstrated elevated levels of polyols (TABLE 1). In addition, one patient with MAN1B1-CDG underwent off-label treatment with epalrestat between the collection of first and second urine samples. This patient's urine demonstrated reduced levels of both sorbitol and mannitol after treatment, suggesting that elevated polyol levels may be a common indicator of many different CDGs, and that ARI treatment can serve to reduce these levels in other CDG patients, not just patients with PMM2-CDG.

### Example 3—Changes in Glycosylation in PMM2-CDG Patients after Epalrestat Treatment

### Methods

Prospectively collected clinical and laboratory data in 24 PMM2-CDG patients: The genetic, laboratory, metabolic, and clinical data of 24 PMM2-CDG patients were evaluated (TABLES 2A and 2B). Disease severity was assessed by Nijmegen Pediatric CDG Rating Scale (NPCRS), (most severe=82; mild (0-14), moderate (15-25), severe (>26)) (Achouitar et al., J Inherit Metab Dis 34:923-927, 2011; and Martinez-Monseny et al., Ann Neurol 85:740-751, 2019). Patient P1 and twin patients P4 and P5 also were discussed elsewhere (Qian et al., JIMD Rep 56:27-33, 2020; and Jaeken et al., Ped Res 14:179, 1980). P1, enrolled in a single IND clinical trial, was additionally evaluated by the International cooperative ataxia rating scale (ICARS) (Trouillas et al., J Neurol Sci 145:205-211, 1997), which assesses limb ataxia, dysarthria, posture and gait disturbances, and oculomotor disorders (0=normal; 100=most severe). Urinary polyols including sorbitol and mannitol were analyzed by GC/MS in 23 out of the 24 PMM2-CDG patients (patient P6 was deceased). In addition, functional in vitro data was collected from the fibroblasts of P1-P6, P8, P10, P17, P19, and P24.

TABLE 1
Polyol levels in various CDG

| | | |
|---|---|---|
| Disorder | Sorbitol | Mannitol |

Type I CDG (protein glycosylation)

| | | |
|---|---|---|
| ALG8-CDG | 8.08 | 22.25* |
| ALG12-CDG | 3.65 | 25.75* |
| SSR4-CDG | 1) 2.37 | 1) 4.49 |
| | 2) 2 | 2) 4 |
| ALG6-CDG | 7.16 | 274.04* |
| ALG6-CDG | 1) 37.22* | 1) 4.78 |
| | 2) 28.72* | 2) 86.72* |
| ALG6-CDG | 1) 1.32 | 1) 5.43 |
| | 2) 4.83 | 2) 3.85 |
| | 3) 2.70 | 3) 10.73 |
| SRD5A3 | 5.17 | 10.59 |
| PGM1-CDG | 3.76 | 9.79 |
| MPI-CDG | 1) 11.07 | 1) 6.92 |
| | 2) 5.21 | 2) 15.94 |

GPI anchor CDG (lipid glycosylation)

| | | |
|---|---|---|
| PIGS-CDG | 1) 8.38 | 1) 40.60* |
| | 2) 16.30* | 2) 5.36 |
| PIGN-CDG | 2.14 | 5.62 |
| PIGN-CDG | 8.57 | 10.00 |
| PIGN-CDG | 4.27 | 11.74 |
| PIGN-CDG | 2.14 | 5.62 |

Type II CDG

| | | |
|---|---|---|
| MOGS-CDG | 20.28* | 198.21* |
| SLC39A8-CDG | 18.0^{‡} | 75.9* |
| SLC39A8-CDG | 1) 4.65 | 1) 7.95 |
| | 2) 21.99* | 2) 35.84* |
| MAN1B1-CDG | 1) 10.44 | 1) 14.92 |
| | 2) 7.222^{‡} | 2) 10.13 |
| MAN1B1-CDG | 3.71 | 18.30* |
| MAN1B1-CDG | 22.18* | 22.31* |
| NANS-CDG | 3.4 | 16.54* |
| CDG type IIx | 15.11* | 27.34* |

*abnormal, elevated polyol excretion
^{‡}post-treatment with epalrestat

TABLES 2A and 2B include data from all patients enrolled for clinical data collection (P1-P24), polyol quantification (P1-P5, P7-P24), included in the in vitro studies (P1-P6, P8, P10, P17, P19, and P24), evaluated for safety and efficacy of epalrestat (P1), and sorbitol and mannitol excretion investigation (P1).

Effect of epalrestat on PM111 enzyme in vitro: Patient-derived and control fibroblasts (GM5381, GM5400; GM5757, GM00038, GM8680, GM01863, GM8400; Coriell Institute, Camden, NJ) were cultured in Minimum Essential Media (MEM; Gibco, Carlsbad, CA; 1 g/L glucose) supplemented with 10% Fetal Bovine Serum (FBS; Gibco), 10% penicillin/streptomycin (P/S) and maintained in an incubator at 37° C., 5% CO₂ in the presence and absence of epalrestat for 24 hours (Iyer et al., Disease Models & Mechanisms 12:11, 2019). Cells were cultured and harvested by trypsinization with 0.05% Trypsin-EDTA (Gibco). PMM and phosphomannose isomerase (MPI) activity were assayed by spectrophotometric measurements (Van Schaftingen and Jaeken, FEBS Lett 377:318-320, 1995). The effects of epalrestat (5 μM, 10 μM, or 20 μM) on PMM-deficient fibroblasts of 11 patients (P1-P6, P8, P10, P17, P19, P24) were evaluated.

Effect of epalrestat on glycosylation biomarkers in vitro: Immunoblotting and RT-qPCR were used to measure Intercellular Adhesion Molecule 1 (ICAM-1) and Lysosomal Associated Membrane Protein 2 (LAMP-2) protein and mRNA expression levels, respectively, as cellular markers of N-glycosylation (Ferrer et al., Mol Genet Metab 131:424-429, 2020; He et al., J Biol Chem 287:18210-18217, 2012; Eskelinen, Mol Aspects Med 27:495-502, 2006; and Radenkovic et al., Mol Genet Metab 132:27-37, 2021) in 10 PMM-deficient fibroblast lines (P1-P6, P8, P17, P19, and P24) and in control fibroblasts, treated with 10 μM epalrestat (the optimal dose based on PMM enzyme activity as described above).

TABLE 2A
General information for 24 individuals with PMM2-CDG

| Patient No. | P1 | P2 | P3 | P4 | P5 | P6 | P7 |
|---|---|---|---|---|---|---|---|
| Pathogenic variant | c.422G > A, | c.422G > A, | c.422G > A, | c.422G > A, | c.422G > A, | c.124A > G, | c.422G > A, |
| | c.415G > A | c.548T > C | c.647A > T | c.338C > T | c.338C > T | c.338C > T | c.357C > A |
| Age/Gender | 6/F | 6/M | 7/M | 44/F | 44/F | 7/M † | 7/M |
| Section I: Current | | | | | | | |
| Function | | | | | | | |
| 1. Vision | ++ | − | − | + | + | +++ | + |
| 2. Hearing | − | − | + | − | − | − | ++ |
| 3. Communication | ++ | + | ++ | ++ | ++ | + | ++ |
| 4. Feeding | − | − | + | + | + | +++ | ++ |
| 5. Self-care | +++ | +++ | ++ | ++ | ++ | +++ | +++ |
| 6. Mobility | +++ | ++ | ++ | +++ | +++ | +++ | +++ |
| 7. Educational | + | + | + | ++ | ++ | ++ | ++ |
| achievement | | | | | | | |
| Section II: System | | | | | | | |
| Specific Involvement | | | | | | | |
| 1. Seizures | − | − | − | − | − | + | +++ |
| 2. Encephalopathy | − | − | − | − | − | + | + |
| 3. Bleeding diathesis | − | + | + | + | + | ++ | − |
| or coagulation defects | | | | | | | |
| 4. Gastrointestinal | − | − | + | + | + | + | − |
| 5. Endocrine | − | − | ++ | + | + | + | − |
| 6. Respiratory | − | − | − | − | − | − | − |
| 7. Cardiovascular | − | − | − | − | + | − | − |
| (over preceding 12 | | | | | | | |
| months) | | | | | | | |
| 8. Renal | − | − | − | − | − | +++ | − |
| 9. Liver | − | − | + | + | + | + | + |
| 10. Blood | − | − | − | − | − | +++ | − |
| Section III: Current | | | | | | | |
| Clinical Assessment | | | | | | | |
| 1. Growth | − | − | + | + | + | − | + |
| 2. Development over | 5 | 5 | 5 | 6 | 5 | 10 | 5 |
| preceding 6 months | | | | | | | |
| 3. Vision with usual | + | + | + | + | + | +++ | + |
| glasses. | | | | | | | |
| 4. Strabismus and eye | + | + | ++ | + | + | + | + |
| movement | | | | | | | |
| 5. Myopathy | ++ | + | + | ++ | ++ | − | + |
| 6. Ataxia | ++ | ++ | ++ | ++ | ++ | +++ | ++ |
| 7. Pyramidal | − | − | − | − | + | − | + |
| 8. Extrapyramidal | − | − | − | − | − | − | ++ |
| 9. Neuropathy | ++ | − | + | ++ | ++ | − | ++ |
| Section I: Current | 11 | 7 | 9 | 11 | 11 | 15 | 15 |
| Function; Total = 21 | | | | | | | |
| Section II: System | 0 | 1 | 5 | 4 | 5 | 13 | 5 |
| Specific Involvement; | | | | | | | |
| Total = 30 | | | | | | | |
| Section III: Current | 13 | 10 | 13 | 15 | 15 | 17 | 15 |
| Clinical Assessment; | | | | | | | |
| Total = 31 | | | | | | | |
| Total = 82; Mild | 24 | 18 | 27 | 30 | 31 | 45 | 35 |
| (0-14), moderate (15-25) | | | | | | | |
| and severe (>26) | | | | | | | |
| Sorbitol | 19.9 | 6.27 | 5.67 | 41 | 41 | N/A | 37 |
| Mannitol | 649 | 5.35 | 122 | 69 | 113 | N/A | 195 |
| Condition | Moderate | Moderate | Severe | Severe | Severe | Severe | Severe |

| Patient No. | P8 | P9 | P10 | P11 | P12 |
|---|---|---|---|---|---|
| Pathogenic variant | c.422G > A, | c.109C > T, | c.98A > C, | c.422G > A, | c.422G > A, |
| | c.203T > G | c.337C > A | c.140C > T | c.722G > C | c.458T > C |
| Age/Gender | 1/M | 2/M | 8/M | 12/M | 16/M |
| Section I: Current | | | | | |
| Function | | | | | |
| 1. Vision | − | − | − | − | − |
| 2. Hearing | − | − | − | − | − |
| 3. Communication | ++ | + | + | + | + |
| 4. Feeding | − | − | − | − | − |
| 5. Self-care | +++ | +++ | ++ | + | + |
| 6. Mobility | ++ | +++ | +++ | + | ++ |
| 7. Educational | ++ | ++ | + | + | ++ |
| achievement | | | | | |
| Section II: System | | | | | |
| Specific Involvement | | | | | |
| 1. Seizures | − | + | − | − | − |
| 2. Encephalopathy | + | − | − | − | − |
| 3. Bleeding diathesis | − | + | + | − | + |
| or coagulation defects | | | | | |
| 4. Gastrointestinal | − | + | − | − | +++ |
| 5. Endocrine | − | − | − | − | − |
| 6. Respiratory | − | − | − | − | − |
| 7. Cardiovascular | − | − | − | − | + |
| (over preceding 12 | | | | | |
| months) | | | | | |
| 8. Renal | − | − | − | − | − |
| 9. Liver | + | + | − | − | − |
| 10. Blood | − | − | + | − | − |
| Section III: Current | | | | | |
| Clinical Assessment | | | | | |
| 1. Growth | + | + | + | − | − |
| 2. Development over | 5 | 5 | 5 | 5 | 5 |
| preceding 6 months | | | | | |
| 3. Vision with usual | − | − | − | − | − |
| glasses. | | | | | |
| 4. Strabismus and eye | + | + | + | − | − |
| movement | | | | | |
| 5. Myopathy | + | ++ | + | + | + |
| 6. Ataxia | + | − | ++ | ++ | ++ |
| 7. Pyramidal | − | − | ++ | − | − |
| 8. Extrapyramidal | + | − | + | − | − |
| 9. Neuropathy | − | − | + | − | + |
| Section I: Current | 9 | 9 | 7 | 4 | 6 |
| Function; Total = 21 | | | | | |
| Section II: System | 2 | 3 | 2 | 0 | 5 |
| Specific Involvement; | | | | | |
| Total = 30 | | | | | |
| Section III: Current | 10 | 9 | 14 | 8 | 9 |
| Clinical Assessment; | | | | | |
| Total = 31 | | | | | |
| Total = 82; Mild | 21 | 21 | 22 | 12 | 20 |
| (0-14), moderate (15-25) | | | | | |
| and severe (>26) | | | | | |
| Sorbitol | 5.11 | 16 | 3 | 3.19 | 3.99 |
| Mannitol | 13.8 | 25 | 6 | 4.65 | 3.64 |
| Condition | Moderate | Moderate | Moderate | Mild | Moderate |

N/A not applicable; + symptom present (+ mild, ++ moderate, +++ severe), − symptom normal/absent, † deceased.
*Abnormal sorbitol and mannitol levels are bolded (sorbitol control <5 mmol/mol creatinine; mannitol control <20 mmol/molcreatinine).

TABLE 2B
General information for 24 individuals with PMM2-CDG

| Patient No. | P13 | P14 | P15 | P16 | P17 | P18 | P19 |
|---|---|---|---|---|---|---|---|
| Pathogenic variant | c.368G > A, | c.647A > G, | c.422 G > A, | c.422 G > A, | c.422G > A, | c.563A > G, | c.422 G > A, |
| | c.722G > C | c.415G > A | c.640-23A > G | c.640-23A > G | c.338C > T | c.691G > A | c.385G > A |
| Age/Gender | 22/F | 70/M | 11/F | 9/M | 7/M | 8/F | 5/M |
| Section I: Current | | | | | | | |
| Function | | | | | | | |
| 1. Vision | − | − | + | + | − | + | − |
| 2. Hearing | − | ++ | ++ | − | − | ++ | − |
| 3. Communication | + | − | − | + | ++ | + | ++ |
| 4. Feeding | − | − | − | + | − | − | ++ |
| 5. Self-care | + | − | ++ | ++ | + | + | ++ |
| 6. Mobility | + | + | ++ | ++ | ++ | ++ | ++ |
| 7. Educational | + | − | ++ | ++ | ++ | ++ | ++ |
| achievement | | | | | | | |
| Section II: System | | | | | | | |
| Specific Involvement | | | | | | | |
| 1. Seizures | − | − | + | ++ | + | − | + |
| 2. Encephalopathy | − | − | − | − | − | − | − |
| 3. Bleeding diathesis | − | − | − | − | + | + | + |
| or coagulation defects | | | | | | | |
| 4. Gastrointestinal | + | + | + | + | − | − | + |
| 5. Endocrine | ++ | − | ++ | − | − | − | ++ |
| 6. Respiratory | − | + | − | + | − | − | − |
| 7. Cardiovascular | − | − | − | − | − | + | ++ |
| (over preceding 12 | | | | | | | |
| months) | | | | | | | |
| 8. Renal | − | − | − | − | − | + | − |
| 9. Liver | − | − | − | − | − | − | + |
| 10. Blood | − | − | − | − | − | − | − |
| Section III: Current | | | | | | | |
| Clinical Assessment | | | | | | | |
| 1. Growth | + | − | − | + | − | − | + |
| 2. Development over | 5 | − | 5 | 5 | 5 | 5 | 5 |
| preceding 6 months | | | | | | | |
| 3. Vision with usual | − | − | + | + | − | + | − |
| glasses. | | | | | | | |
| 4. Strabismus and eye | − | + | ++ | + | + | − | ++ |
| movement | | | | | | | |
| 5. Myopathy | + | − | + | + | + | + | − |
| 6. Ataxia | + | ++ | ++ | ++ | ++ | ++ | +++ |
| 7. Pyramidal | − | − | − | − | − | + | − |
| 8. Extrapyramidal | − | − | − | + | − | + | − |
| 9. Neuropathy | − | − | ++ | ++ | + | + | − |
| Section I: Current | 4 | 3 | 9 | 9 | 7 | 9 | 10 |
| Function; Total = 21 | | | | | | | |
| Section II: System | 3 | 2 | 4 | 4 | 2 | 3 | 8 |
| Specific Involvement; | | | | | | | |
| Total = 30 | | | | | | | |
| Section III: Current | 8 | 3 | 13 | 15 | 10 | 11 | 11 |
| Clinical Assessment; | | | | | | | |
| Total = 31 | | | | | | | |
| Total = 82; Mild | 15 | 8 | 26 | 28 | 19 | 23 | 29 |
| (0-14), moderate (15-25) | | | | | | | |
| and severe (>26) | | | | | | | |
| Sorbitol | 2.24 | 2.8 | 5.86 | 11.5 | 5.16 | 10 | 14.8 |
| Mannitol | 4.75 | 7.99 | 22.5 | 119 | 6.96 | 14 | 28.5 |
| Condition | Moderate | Mild | Severe | Severe | Moderate | Moderate | Severe |

| Patient No. | P20 | P21 | P22 | P23 | P24 |
|---|---|---|---|---|---|
| Pathogenic variant | c.422G > A, | c.422G > A, | c.422G > A, | c.422G > A, | c.422G > A, |
| | c.357C > A | c.385G > A | c.691G > A | c.713G > A | c.623G > C |
| Age/Gender | 3/M | 4/F | 4/M | 8/M | 10/F |
| Section I: Current | | | | | |
| Function | | | | | |
| 1. Vision | − | − | + | + | − |
| 2. Hearing | − | − | − | − | − |
| 3. Communication | ++ | ++ | ++ | + | ++ |
| 4. Feeding | ++ | − | ++ | ++ | +++ |
| 5. Self-care | +++ | +++ | +++ | − | +++ |
| 6. Mobility | ++ | ++ | +++ | − | +++ |
| 7. Educational | + | ++ | ++ | − | ++ |
| achievement | | | | | |
| Section II: System | | | | | |
| Specific Involvement | | | | | |
| 1. Seizures | − | + | + | − | − |
| 2. Encephalopathy | − | − | − | − | − |
| 3. Bleeding diathesis | + | + | + | − | + |
| or coagulation defects | | | | | |
| 4. Gastrointestinal | + | − | + | ++ | − |
| 5. Endocrine | ++ | − | − | ++ | + |
| 6. Respiratory | − | − | − | − | − |
| 7. Cardiovascular | − | + | − | − | − |
| (over preceding 12 | | | | | |
| months) | | | | | |
| 8. Renal | − | − | − | − | − |
| 9. Liver | + | − | + | − | − |
| 10. Blood | + | − | − | + | ++ |
| Section III: Current | | | | | |
| Clinical Assessment | | | | | |
| 1. Growth | + | + | + | + | +++ |
| 2. Development over | 5 | 5 | 5 | 5 | 6 |
| preceding 6 months | | | | | |
| 3. Vision with usual | − | − | + | + | − |
| glasses. | | | | | |
| 4. Strabismus and eye | + | + | − | − | ++ |
| movement | | | | | |
| 5. Myopathy | + | − | ++ | − | ++ |
| 6. Ataxia | ++ | ++ | ++ | + | +++ |
| 7. Pyramidal | − | − | ++ | − | − |
| 8. Extrapyramidal | + | + | ++ | − | − |
| 9. Neuropathy | + | + | + | − | − |
| Section I: Current | 10 | 9 | 13 | 5 | 13 |
| Function; Total = 21 | | | | | |
| Section II: System | 6 | 3 | 4 | 5 | 4 |
| Specific Involvement; | | | | | |
| Total = 30 | | | | | |
| Section III: Current | 12 | 11 | 16 | 9 | 16 |
| Clinical Assessment; | | | | | |
| Total = 31 | | | | | |
| Total = 82; Mild | 28 | 23 | 33 | 19 | 33 |
| (0-14), moderate (15-25) | | | | | |
| and severe (>26) | | | | | |
| Sorbitol | 13.6 | 8.61 | 19.7 | 7.47 | 2.69 |
| Mannitol | 97.5 | 35.5 | 21.1 | 33.5 | 7.03 |
| Condition | Severe | Moderate | Severe | Moderate | Severe |

N/A not applicable; + symptom present (+ mild, ++ moderate, +++ severe), − symptom normal/absent, † deceased.
*Abnormal sorbitol and mannitol levels are bolded (sorbitol control <5 mmol/mol creatinine; mannitol control <20 mmol/mol creatinine).

Proteomics and glycoproteomics: Cell were scraped in PBS, pH 7.4 and sonicated with a tip sonicator at 40% amplitude for 3 cycles of 10 seconds each. Equal amount of proteins were digested with trypsin as described elsewhere (Mun et al., Anal Chem 92:14466-14475, 2020). The digested peptides were labeled with tandem mass tag (TMT) reagents as per the manufacturer's instructions (ThermoFisher). Labelled samples were pooled, and either size-exclusion chromatography or basic pH reversed-phase fractionation was performed. An aliquot of dried peptides was resuspended in 100 μL of 0.1% formic acid and injected into Superdex peptide 10/300 column (GE Healthcare). Twenty-one (21) early fractions were collected starting at 10 minutes after injection (total run time of 130 minutes) and analyzed by LC-MS/MS as described elsewhere (Mun et al., supra). Another aliquot of total peptides was cleaned up by C18 TopTips (Glygen) and fractionated by bRPLC on a reversed phase C18 column (4.6×100 mm column). Twelve (12) fractions were dried and re-suspended in 0.1% formic acid for LC-MS/MS analysis.

A modification of the LC-MS/MS parameters described by Mun et al. (supra) were used. Specifically, 21 early fractions from SEC and 12 fractions of bRPLC were analyzed by Orbitrap Exploris480 mass spectrometer (Thermo Fisher Scientific) coupled to an EASY-Spray column (Thermo Fisher Scientific). Every run was 130 minutes with a flow rate of 300 nL/minute. The gradient used for separation was: equilibration at 3% solvent B from 0 to 4 minutes, 3% to 10% sol B from 4 to 10 minutes, 10% to 35% sol B from 10.1 to 125 minutes, and 35% to 80% sol B from 125 to 145 minutes. All experiments were done in DDA mode with the top 15 ions isolated at a window of 0.7 m/z and a default charge state of +2. Charge states ranging from +2 to +7 were considered for MS/MS events. Stepped collision energy was applied to precursors at normalized collision energies of 15, 25, and 40. The MS precursor mass range was set to 375 to 2000 m/z and 100 to 2000 for MS/MS. The automatic gain control for MS and MS/MS were 1×10⁶ and 1×10⁵ and the injection times to reach AGC were 50 ms and 250 ms, respectively. 60 second dynamic exclusion was applied. Data acquisition was performed with the option of Lock mass (441.1200025 m/z) for all data.

Database searching and analysis: The publicly available software pGlyco Version 2.2.0 was used (Zeng et al., Sci Rep 6:25102, 2016; and Liu et al., Nat Commun 8:438, 2017). A glycan database containing 8,092 entries and Uniprot Human Reviewed protein sequences (20,432 entries) were used as protein sequence fasta files. Cleavage specificity was set to fully tryptic with 2 missed cleavages, and precursor and fragment tolerance were set to 5 and 20 ppm. Cysteine carbamidomethylation was set as fixed modification, and oxidation of methionine was set as variable modification. The results were filtered to retain only entries that had 1% FDR at the glycopeptide level. Reporter ion quantification was performed in Proteome Discoverer 2.5 using “reporter ion quantifier” node and Ids were matched with quantitation on a scan-to-scan basis (MS/MS). A proteomics dataset was searched using Sequest in Proteome Discoverer 2.4.

Correlation analysis between polyol levels and CDG disease severity, including neuropathy: The correlation between urine sorbitol and mannitol levels and the severity of NPCRS, patient age, growth, the degree of glycosylation abnormality was assessed based on transferrin glycoform analysis, organ-specific scores for liver involvement, and severity of the neuropathy according to NPCRS.

### Phase I Study of Epalrestat in a Single Patient with PMM2-CDG

Study design: An open label, single patient (P1) compassionate use study was designed to assess the safety and tolerability of oral epalrestat therapy in a child with PMM2-CDG.

Patient assessment: Prior to the first dose of epalrestat and at 1, 2, 3, 6, 8, 9, 12 months during treatment, concomitant medications and vital signs were recorded and the patient was evaluated using the NPCRS. In addition, blood was drawn for serum chemistry, hematology, and plasma levels of epalrestat. Urine was collected for polyol measurement at baseline and twice over the course of 12 months of therapy.

Drug administration: Epalrestat was administered orally, 3 times per day (TID) before meals in a divided dose (0.8 mg/kg/day; 5 mg TID) of epalrestat (Ono Pharmaceuticals, Osaka, Japan) starting on Day 1 of the study.

Safety and efficacy: Safety measures included vital signs, change in CBC, liver function (INR, bilirubin, transaminases, and albumin) in blood, change in liver elastography, and following the pharmacokinetic profile of epalrestat. The efficacy of epalrestat was studied by clinical follow up (physical exam, NPCRS, ICARS), standard laboratory tests, and transferrin glycoform analysis by mass spectrometry.

Pharmacokinetics: Epalrestat plasma concentrations were measured using an LC-MS/MS assay that was validated according to principles outlined in FDA Guidance Documents. Epalrestat-d₅ was utilized as the internal standard. The mass spectrometer was coupled to a Waters Acquity H class ultra-performance liquid chromatography system (Milford, MA). Data were acquired and analyzed with Waters MassLynx v4.1 software. The chromatographic separation of epalrestat and the internal standard was accomplished using an Agilent Infinity Lab Poroshell 120 EC-C18 column, 2.1×100 mm, 2.7 μm (ChromTech, Apple Valley, MN) with an Agilent Poroshell 120 EC-C18 precolumn, 2.1×5 mm, 2.7 μm (ChromTech). Plasma samples were analyzed after protein precipitation with methanol, concentration to dryness under nitrogen, and reconstitution in methanol:water (1:1 v/v). Pharmacokinetics were estimated by non-linear least squares regression using the program Phoenix WinNonlin.

Urine sample collection: Urine was collected for polyol measurement at baseline and twice over the course of 12 months of therapy.

Statistical analyses: All data are expressed as mean±SD. Statistical analyses were performed using either GraphPad Prism 8.3 or JAMOVI v.1.6.9 (online at jamovi.org). Two sample t tests were used to compare two groups, while one-way ANOVA was used to compare groups of three or more. Shapiro Wilk test was used for normality. Non-parametric tests (Mann Whitney test for comparing two groups and Kruskal-Wallis test for three or more groups) were used if dependent variables were not normally distributed. p-value <0.05 was considered significant. Bonferroni p-value correction was applied for multiple comparisons. p<0.05(*), p<0.01(**), p<0.001(***), p<0.0001(****). NPCRS scores were collapsed into three categories of mild, moderate, and severe, and correlation coefficients with mannitol and sorbitol measurements were calculated using Pearson's r (p<0.05). Liver involvement, already in the form of binary values, was correlated with the same approach (p<0.05).

### Results

Prospectively collected clinical data: In the cohort of 16 males and 8 females, the mean age was 13 years, (range 1 to 70 years, one male patient (P6) deceased at the age of 7). Nineteen patients were younger than 16 years old, and four were adults. Transferrin glycoform ratios were available for 23 patients. The total median NPCRS score of all patients was 23 (moderate phenotype), ranging from 8 to 35, with 2 mild, 11 moderate, and 11 severe phenotypes. The most common genotypes were c.422G>A/c.357C>A in three patients, c.422G>A/c.338C>T in three patients and c.422 G>A/c.385G>A in two patients. Overall, the most common pathogenic variant was c.422G>A (15/24), followed by c.338C>T (4/24) and c.357C>A (3/24)

Neurological symptoms were the most frequent findings, as almost all patients (23/24) presented with severe developmental disability and cerebellar ataxia. Most suffered from hypotonia (19/24), neuropathy (14/24), or a movement disorder (8/24), leading to impaired mobility (23/24) and communication skills (22/24). Almost half of the patients (10/24) suffered from seizures and (mostly mild) visual impairment (10/24). Over half of the patients (18/24) had strabismus. Hearing loss (5/24), spasticity (5/24), and encephalopathy (3/24) were relatively rare. About half of the patients had either coagulation abnormalities (15/24), gastrointestinal symptoms (14/24), endocrine disturbances (10/24), and/or liver involvement (10/24) (TABLES 2A and 2B).

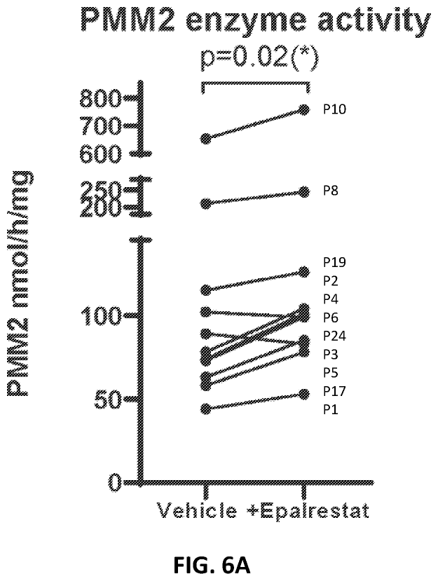

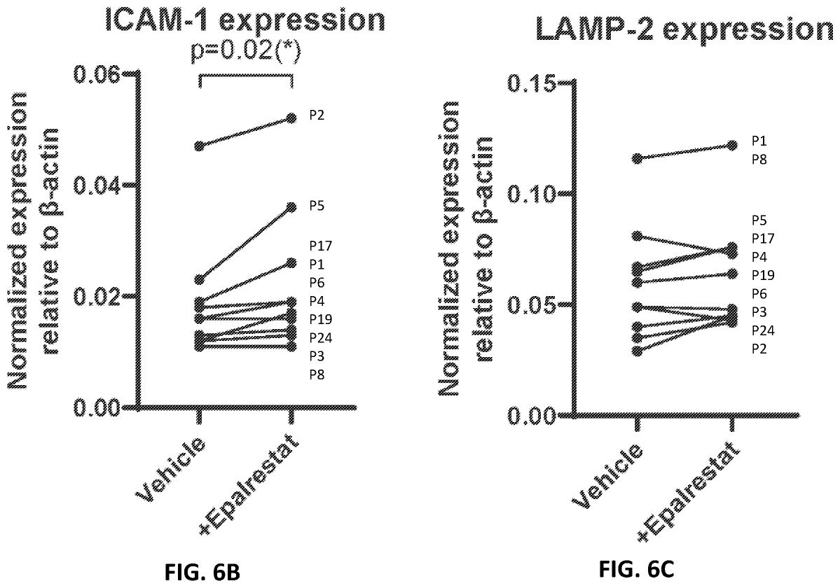

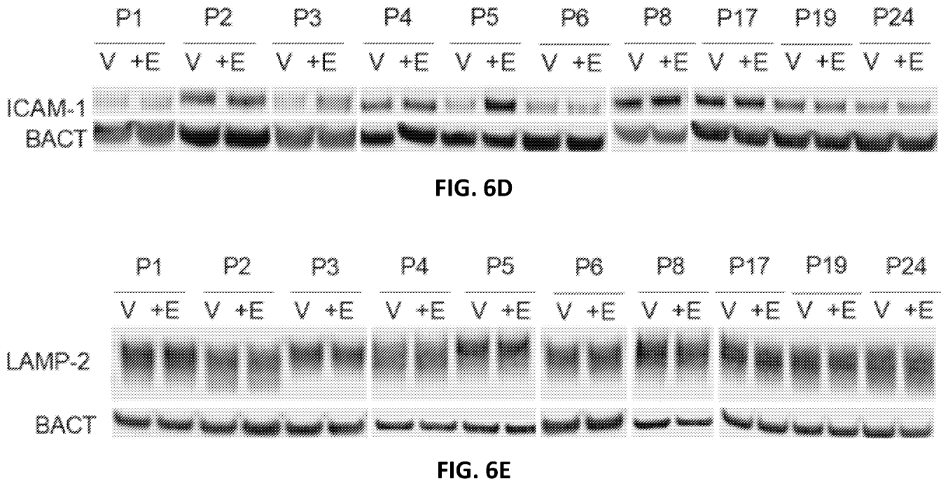

Epalrestat increases PMM enzyme activity in vitro: Nine out of ten patients showed at least a 10% increase in PMM enzyme activity with 5 μM, 10 μM or 20 μM epalrestat treatment, exhibiting up to a 50% increase in PMM activity with at least one of the three concentrations. Three patients exhibited the highest PMM enzyme activity (nmol/h/mg) when treated with 5 μM epalrestat (43% in P1, 35% in P5, and 22% in P10); four exhibited the highest PMM enzyme activity with 10 μM epalrestat (33% in P2, 35% in P3, 16% in P8, and 36% in P24); and three patients showed improved PMM enzyme activity at 20 μM epalrestat (50% in P4, 10% in P17, 20% in P19). Altogether, 80% (8/10) of patient fibroblasts responded to 10 μM epalrestat with a 10% to 36% increase in PMM enzyme activity (FIG. 6A and TABLE 3). It is noted that a 10 μM concentration of epalrestat is comparable to the concentration measured in human blood after epalrestat treatment.

TABLE 3
Epalrestat treatment of fibroblasts from PMM2-CDG individuals

| | | Fold | Fold | Fold | | | |
|---|---|---|---|---|---|---|---|
| | | change | change | change | | | Highest |
| Subject | Base- | 5 μM | 10 μM | 20 μM | Best | | fold |
| (P = 11) | line | epalrestat | epalrestat | epalrestat | dose | | change |
| P1 | 1 | 1.43 | 1.2 | 1.2 | 5 | μM | 1.43* |
| P2 | 1 | 1.01 | 1.33 | 0.7 | 10 | μM | 1.33 |
| P3 | 1 | 0.86 | 1.35 | 0.9 | 10 | μM | 1.35 |
| P4 | 1 | 1.26 | 1.36 | 1.5 | 10 | μM | 1.36 |
| P5 | 1 | 1.35 | 0.93 | 0.9 | 5 | μM | 1.35 |
| P6 | 1 | 0.98 | 0.97 | 0.6 | — | | — |
| P8 | 1 | 1.07 | 1.16 | 1.1 | 10 | μM | 1.16 |
| P10 | 1 | 1.22 | 1.16 | 1.2 | 5 | μM | 1.22 |
| P17 | 1 | 0.9 | 0.75 | 1.1 | 20 | μM | 1.1 |
| P19 | 1 | 0.84 | 1.1 | 1.2 | 20 | μM | 1.2 |
| P24 | 1 | 0.78 | 1.36 | 0.9 | 10 | μM | 1.36 |

*The highest fold changes are bold.

Effect of epalrestat on classical glycosylation biomarkers: ICAM-1 and LAMP-2 mRNA expression in PMM2-deficient and control fibroblasts were similar. Treatment with 10 μM epalrestat had no effect on either ICAM-1 or LAMP-2 mRNA expression.

Western blot analysis of PMM-deficient fibroblasts (n=10; P1-P6, P8, P17, P19, and P24) revealed that although ICAM-1 was decreased in some individuals, no significant difference was observed in the abundance of ICAM-1 compared to controls. Treatment with 10 μM epalrestat resulted in an increase in ICAM-1 protein expression (p=0.02; FIGS. 6B and 6D). LAMP-2 protein abundance was comparable between PMM-deficient and control fibroblasts before treatment, but all patient fibroblasts showed a “smear pattern” resulting from differences in LAMP2 mass due to decreased glycosylation, compared to a single LAMP-2 band in observed in controls (FIG. 6E). LAMP-2 protein abundance showed a marginal but not significant increase after treatment (FIG. 6C), and no change in the “smear pattern” (FIG. 6E).

PMM-deficient fibroblasts exhibit moderate reduction of selected proteins and global reduction in N-glycosylation: Six patient-derived fibroblast populations (P1-P6) were treated with epalrestat, and paired samples as well as four untreated control fibroblast populations were analyzed by deep multiplexed proteomics and glycoproteomics. 6,636 proteins with 61,124 peptides were identified and quantified. In addition, the abundance of 6,061 individual intact glycopeptides with 249 unique glycan compositions (554 glycan structures) on 926 glycosylation sites of 494 glycoproteins was established.

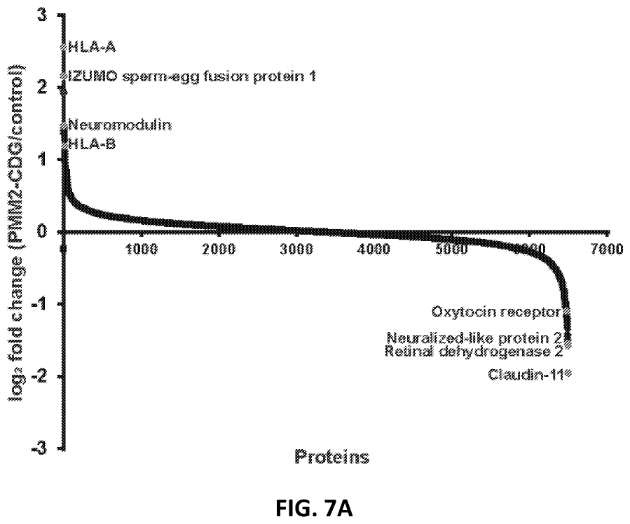

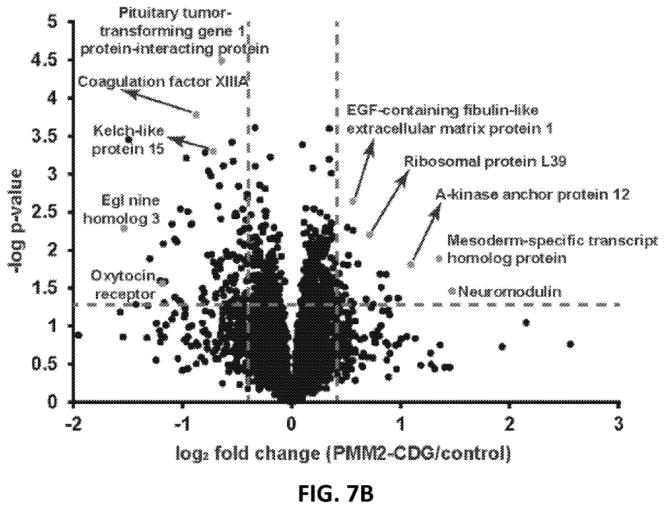

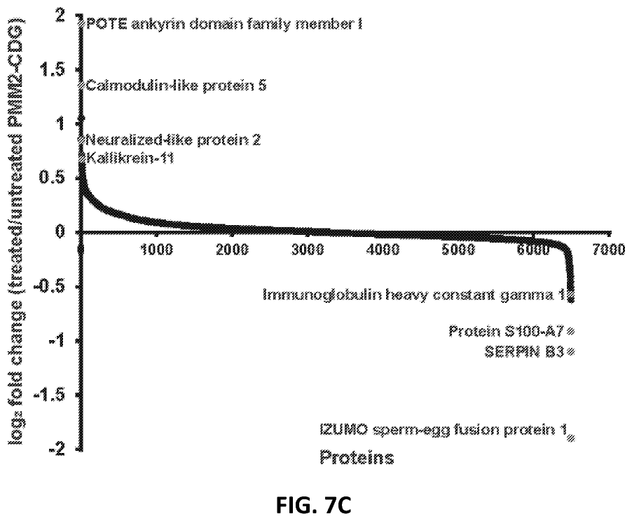

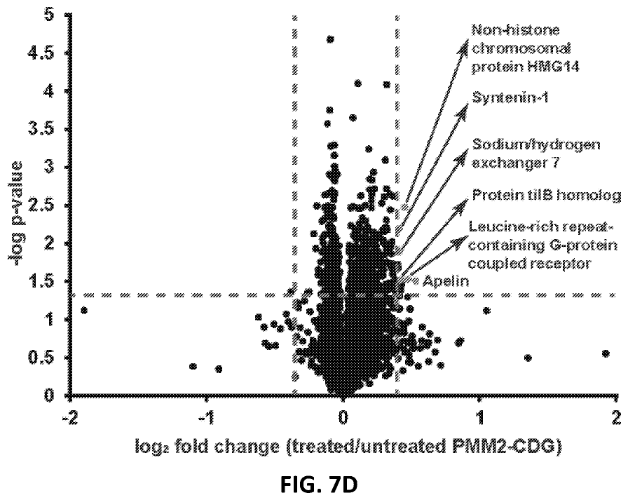

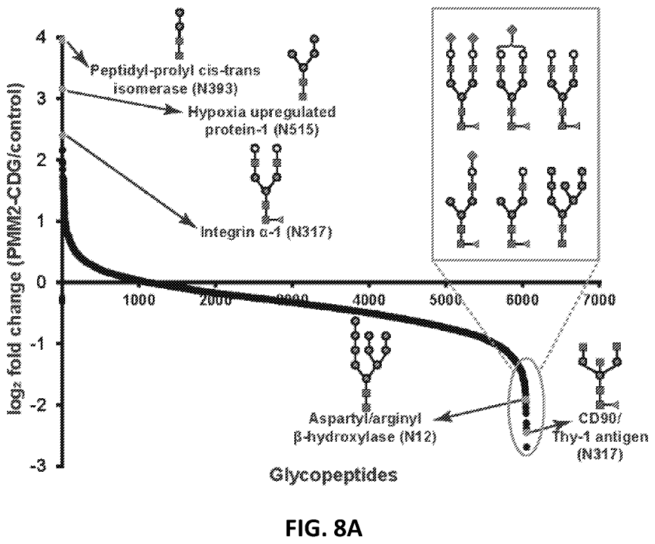

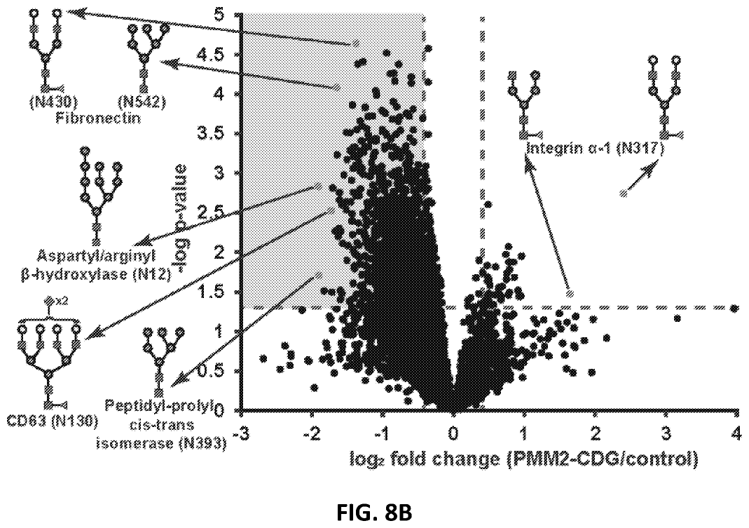

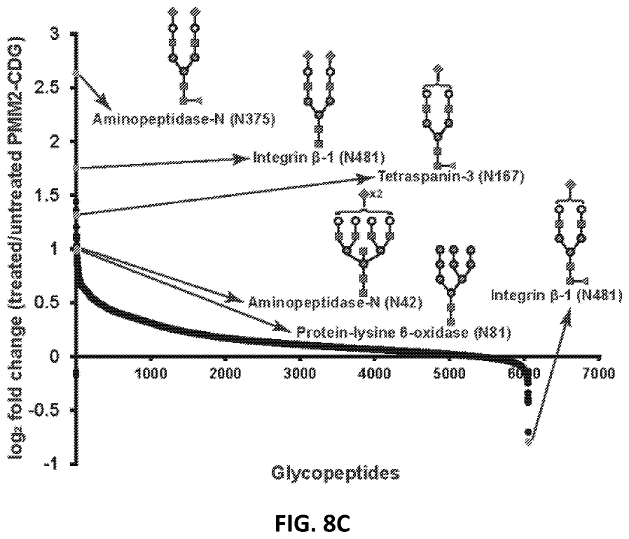

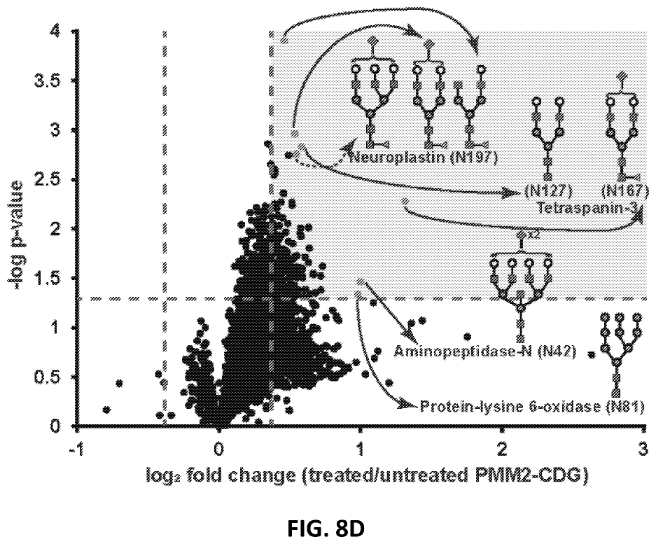

In untreated PMM-deficient fibroblasts, 563 proteins were significantly different from controls. PMM2 protein levels were reduced 1.6-fold in PMM-deficient fibroblasts compared to controls, while mannose-6-phosphate isomerase was not different. Globally, after imposing a 30% change cutoff (fold change 1.3) as a filter, 147 proteins remained different, of which 102 were reduced in abundance (FIGS. 7A and 7B). Only 15 proteins had two-fold or more reduced abundance, and 3 proteins were increased two-fold. Among the notable reduced proteins, prolyl hydroxylase EGLN3, vascular endothelial growth factor receptor 3, fibrinogen beta and gamma chains, and tissue factor were identified. The glycoproteome had widespread alterations in PMM-deficient fibroblasts (FIGS. 8A and 8B), and differential abundance of 1,497 glycopeptides was found, of which 1,448 were reduced compared to controls (p<0.05). The global reduction in N-glycosylation is visible in FIGS. 8A and 8B, in which the volcano plot (FIG. 8B) is skewed to the left. Out of 1,448 reduced glycopeptides, a 30% change cutoff qualified 1,289 glycopeptides as being significantly reduced. The glycopeptide with the greatest reduction was aspartyl/asparaginyl beta-hydroxylase (N12, Hex10HexNAc2), which is a calcium sensor in the endoplasmic reticulum-plasma membrane junction. The most affected glycoproteins with global reduction in glycosylation were fibronectin (FN, 78 glycopeptides from 5 glycosylation sites), basement membrane-specific heparan sulfate proteoglycan core protein (HSPG2, 17 glycopeptides from 3 sites), protein-lysine 6-oxidase (LYOX1, 17 glycopeptides from one site, N81), Prolow-density lipoprotein receptor-related protein 1 (LRP1, 14 glycopeptides from 5 sites), CD63 (13 glycopeptides from 1 site, N130), and CD166 (13 glycopeptides from 3 sites). Notably, 36 glycopeptides of LAMP-1 and 16 glycopeptides of LAMP-2 were also markedly reduced. ICAM-1 protein levels were not significantly different between PMM-deficient fibroblasts and controls by proteomics measurements. In addition, seven complex type glycans were detected at Asn267 of ICAM-1 protein, which followed the trend of not being significant between the PMM-deficient fibroblasts and controls.

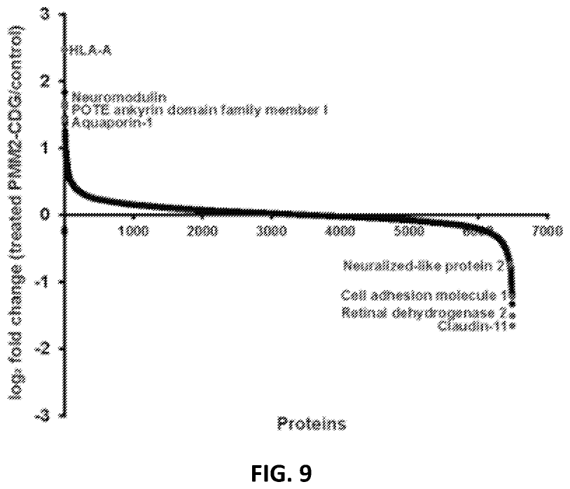

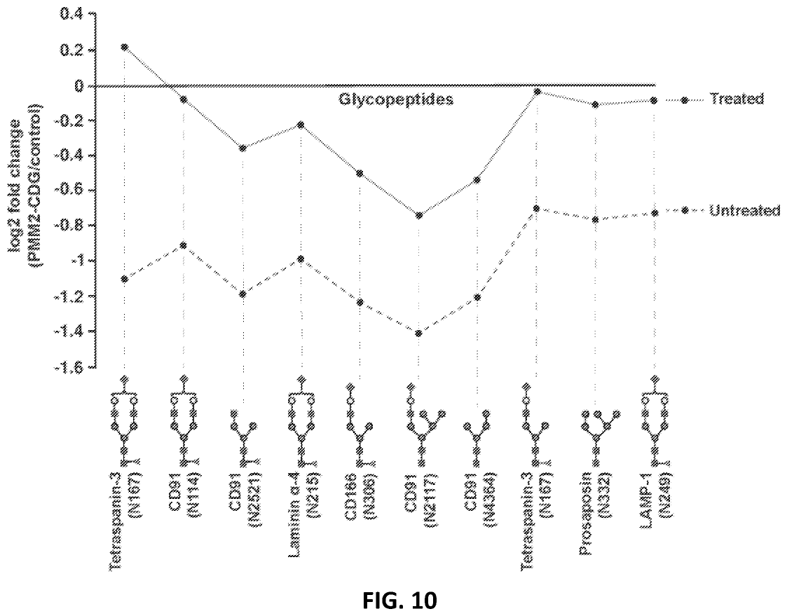

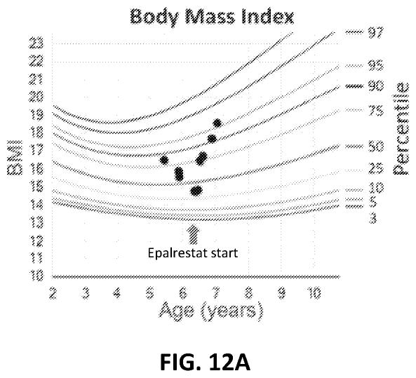

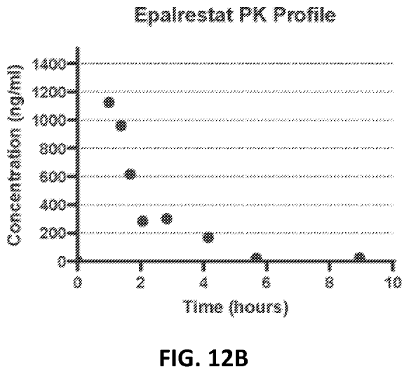

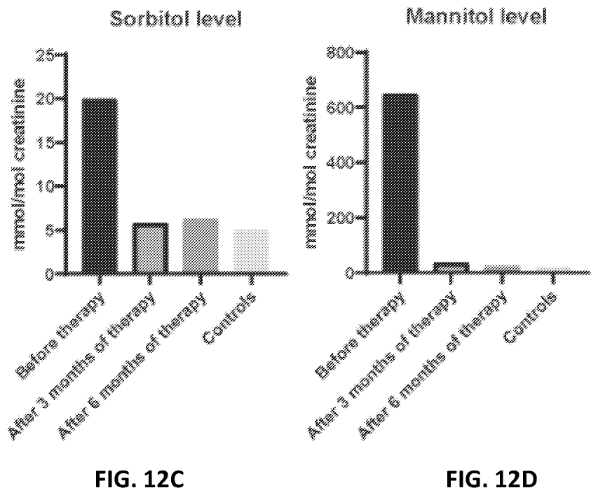

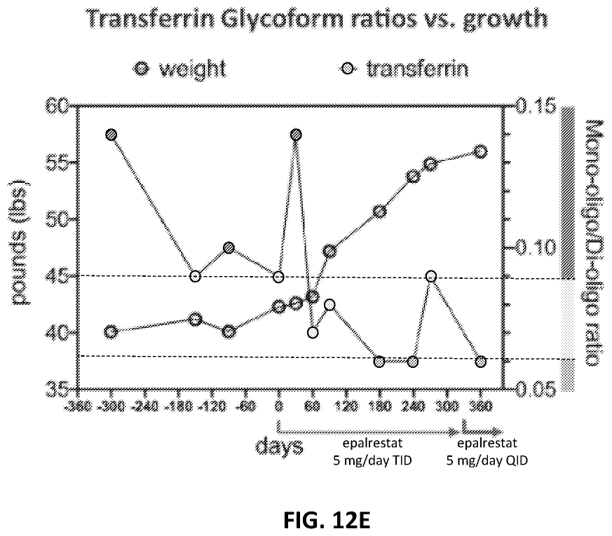

Epalrestat treatment improves global glycosylation profile of PW deficient fibroblasts: In paired sample analysis before and after epalrestat treatment of PMM2-CDG patient fibroblasts, proteomic measurements revealed 628 proteins to be different post-treatment, which was reduced to only 13 proteins with a 30% or bigger change cutoff (FIGS. 7C and 7D), 12 of which were increased post treatment. Untreated and treated fibroblasts did not show marked changes in protein levels (FIGS. 7A and 9). Sorbitol dehydrogenase (SORD) showed a modest 11% increase in abundance, but the reduced PMM2 protein levels did not improve upon epalrestat treatment in any comparison (FIG. 7C). Looking at the glycoproteome, 412 glycopeptides had differential abundance, with none of them reduced in epalrestat-treated fibroblasts (FIGS. 8C and 8D). These 412 glycopeptides, which significantly improved in their abundance upon epalrestat treatment (FIG. 8D), also included 97 glycopeptides that had reduced glycosylation in PMM-deficient fibroblasts compared to controls. Twenty four of these 97 glycopeptides contained high-mannose glycans (Man4-Man9). When treated fibroblasts were compared to untreated controls, 665 of 1,448 glycopeptides (46%) that had reduced abundance in untreated PMM-deficient fibroblasts (PMM2 vs. controls) were improved post-treatment, becoming similar to controls. About a third (217) of these glycopeptides contained Man4-Man9 glycans, while others had complex/hybrid type glycan structures. One of the complex type glycans was at Asn267 of ICAM-1 protein, which was highly fucosylated at the branch in addition to being core-fucosylated (Hex7HexNAc6NeuAc2Fuc5 at Asn267), was modestly increased (10%) in abundance in paired epalrestat vs. vehicle analysis. Among the glycoproteins that became similar to controls in glycopeptide abundance upon treatment, CD63, CD166, LRP1, LAMP-1, LAMP-2, LYOX1, FN, alpha- and beta-integrins, collagen family members, and CD44 were notable. The glycopeptides that showed the greatest improvements are indicated in FIG. 10.

Elevated urine sorbitol and mannitol in PMM2-CDG patients with peripheral neuropathy and liver pathology: Urine polyol levels (erythritol, arabinitol, ribitol, galactitol) were normal except for sorbitol and mannitol in most PMM2-CDG patients. Urine sorbitol levels ranged from 2.24 to 41 mmol/mol creatinine (controls; <5 mmol/mol creatinine) and 74% of the PMM2-CDG patients presented with an increased urine sorbitol level (17/23) (TABLE 2). Urine mannitol levels ranged from 3.64 to 648.6 mmol/mol creatinine (controls; <20 mmol/mol creatinine), with 61% of the PMM2-CDG patients presenting with increased urine mannitol level (14/23) (TABLE 2).

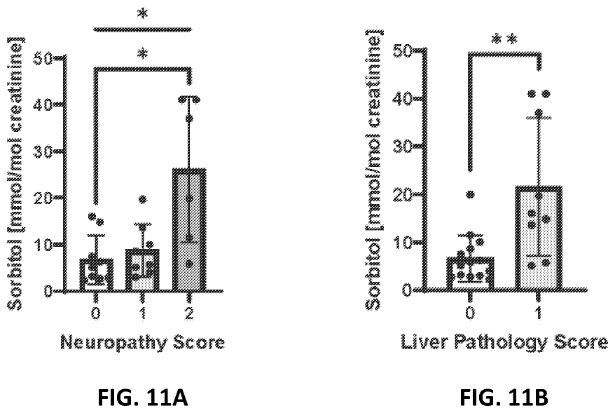

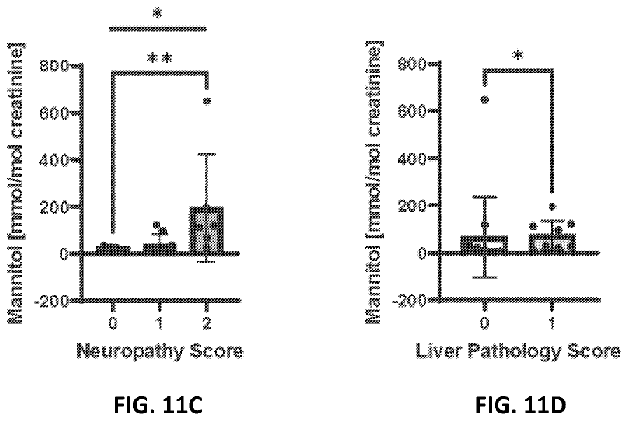

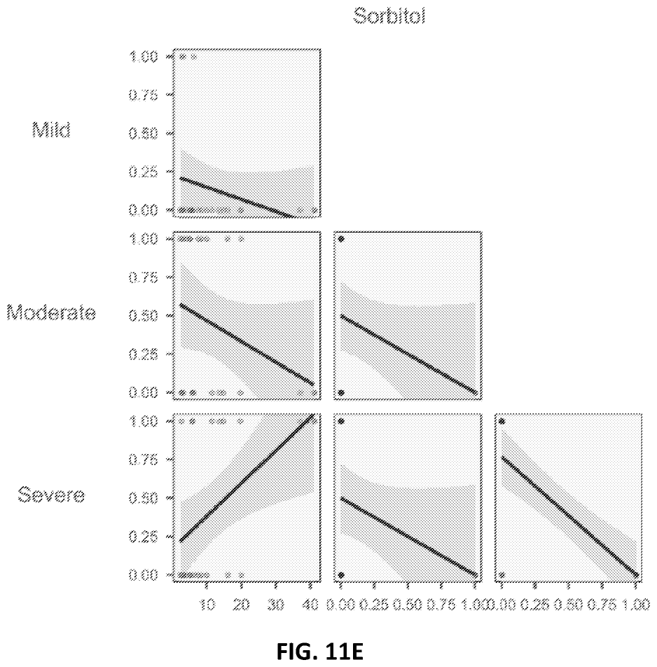

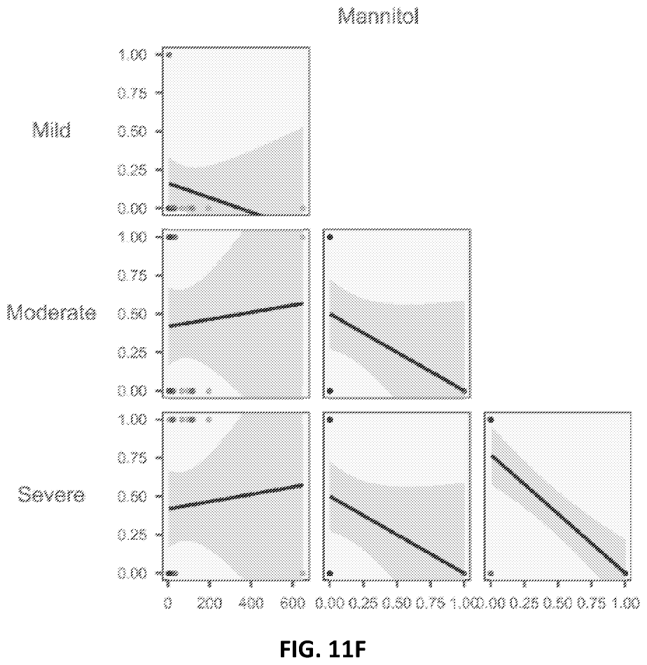

Urine concentrations of sorbitol (p=0.015) and mannitol (p=0.001) were higher in patients with moderate peripheral neuropathy, compared to no neuropathy (FIGS. 11A and 11C). Urine sorbitol (p=0.004) and mannitol (p=0.02) also were increased in patients with mild liver pathology (elevated transaminases; FIGS. 11B and 11D). Urine sorbitol levels were positively correlated (r=0.5) with “severe” neuropathy (p<0.02), but not with mild or moderate neuropathy (patient category based on NPCRS severity scores; FIG. 11E). Mannitol measurements did not correlate with any categories (FIG. 11F). No significant correlation was observed between urine sorbitol or mannitol levels and age, growth, or mono/di-oligo and a-oligo/di-oligo transferrin levels.

Safety and efficacy of epalrestat in a single PMM2-CDG patient: The P1 single-dose PK data on 0.27 mg/kg epalrestat three times a day (0.8 mg/kg/day or 15 mg/day; a third of the epalrestat dose used in diabetic adults) were comparable to doses successfully used in the culture media of patient fibroblasts. No adverse events were reported. All vital signs remained normal throughout the duration of the study. Standard Laboratory Screening was performed; values for CBC with differential (a full blood count), transaminases, bilirubin, and albumin remained normal throughout the study. In addition, antithrombin III levels and International normalized ratio (INR) remained at normal levels throughout the study. Liver elastography remained normal. The Interquartile Range to Median ratio prior to therapy was 14%, after 1 month of therapy was 12%, at 6 months was 20%, and at 12 months was at 8% (normal is below 25%).

Efficacy across multiple outcome measures: The epalrestat-treated patient's ICARS score improved from a score of 56 to a score of 42 within 12 months. Prior to enrollment, the patient was under treatment for five months with acetazolamide (AZA; Martinez-Monseny et al., Ann Neurol 85:740-751, 2019), which produced an improvement in the ICARS score from 66 to 56 before the start of epalrestat treatment. AZA was discontinued for one month prior to the start of epalrestat dosing. After withdrawal of AZA, patients typically regress to pre-intervention scores within 5-8 weeks (Martinez-Monseny et al., supra). However, treatment with epalrestat not only prevented the expected reversal, but it showed further improvement to an ICARS score of 42.

The body mass index (BMI) of the patient showed a notable improvement without any diet modification, increasing to 18.5 (95th percentile) from its previous trough at 14.8 (30th percentile). This mirrored the patient's improved appetite and potentially improved absorption in a 12-month-follow-up period (FIG. 12A).

The NPCRS indicated a minimal improvement from a baseline between 21-24 in the six month period before the trial to a score of 20-21 between months 6, 9, and 12.

The level of blood transferrin glycosylation (Mono-oligo:Di-oligo ratio) showed a significant improvement after treatment. Before treatment, the level was abnormal and ranged from 0.09 to 0.14 (normal≤0.06). After 6 months of therapy, the level of transferrin had normalized (0.06 at 6 months). At the 9 month visit, transferrin glycosylation had become marginally abnormal (0.09 at 9 months), but the patient was on a suboptimal epalrestat dose due to weight gain. The transferrin normalized with dose correction (0.06 at 12 months) (FIG. 12E and TABLE 4).

A pharmacokinetic profile of epalrestat was conducted for patient P1 (administered a 0.27 mg/kg epalrestat dose three separate times). The plasma epalrestat concentration over time following an oral dose of epalrestat showed rapid absorption and elimination, mirroring the rapid elimination observed in adults (FIG. 12B). A peak concentration of 1125 ng/ml (3.5 μM) epalrestat in plasma occurred one hour after the epalrestat administration. The trough level as the lowest concentration reached by epalrestat before the next dose was 23.4 ng/ml (0.1 μM) after eight hours. Epalrestat was eliminated with a t½ of about 1.04 hours. The systemic exposure (AUC) and oral clearance after a 5 mg dose of epalrestat were 2792 hr*ng/mL and 1.8 L/hr, respectively.

Urine sorbitol and mannitol levels were significantly elevated before therapy in the PMM2-CDG patient as compared to controls. As shown in FIGS. 12C and 12D, epalrestat treatment nearly normalized urine sorbitol and mannitol levels compared to controls. Improvements in urine sorbitol levels were observed in parallel with biochemical and clinical improvements (FIG. 12E). Other polyols (erythritol, arabitol, ribitol, galactitol) were normal.

TABLE 4
Blood transferrin glycoform ratio analysis
Carbohydrate deficient transferrin for CDG

| Time of sampling | Mono-oligo/Di-oligo | A-oligo/Di-oligo |
|---|---|---|
| 1 year prior to trial | 0.14 | 0.01 |
| 6 months prior to trial | 0.09 | 0.003 |
| 3 months prior to trial | 0.10 | 0.007 |
| Prior to first dose | 0.09 | 0.005 |
| 1st month | 0.14 | 0.005 |
| 2nd month | 0.07 | 0.01 |
| 3rd month | 0.08 | 0.004 |
| 6th month | 0.06 | 0.003 |
| 8th month | 0.06 | 0.004 |
| 9th month | 0.09* | 0.004 |
| 12th month | 0.06 | 0.004 |

Mono-oligo/Di-oligo control: ratio ≤ 0.06
A-oligo/Di-oligo control: ratio ≤ 0.01
*Patient was underdosed due to unexpected weight gain, with dose correction to 0.8 mg/kg/day at 11 months.
Abnormal values are in bold.

### Example 4—Changes in MS-Determined Protein Glycosylation in Patient Fibroblasts Deemed Responsive and Non-Responsive by Enzymatic Assay

As shown in FIG. 8D, 412 glycopeptides showed enhanced abundance levels (highlighted box) after epalrestat treatment. Each individual solid circle represents one glycopeptide.

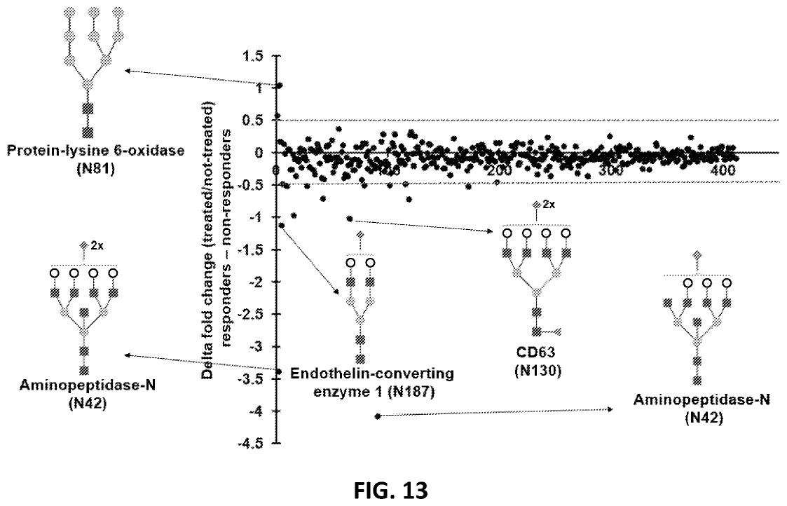

To compare responders (patients who had more than a 20% increase in enzyme activity after epalrestat treatment) to non-responders (patients who had less than a 20% increase in enzyme activity after epalrestat treatment) and gauge the increase in glycosylation upon epalrestat treatment, linear fold changes (treated/untreated) were calculated for each of these 412 glycopeptides. The fold changes of non-responders were subtracted from the fold changes of the responders, and the fold change difference (delta fold change) was calculated. These delta fold changes were plotted (FIG. 13, Y-axis) to show the difference in the glycosylation increase at the glycopeptide level between responders and non-responders. A negative delta fold change value indicated that the relative abundance of these glycopeptides was increased more in non-responders than in responders, while a positive delta fold change value showed that responders had a higher increase in glycosylation levels than non-responders. The closer to zero the delta fold change value (indicated by the solid horizontal line in FIG. 13), the less difference there was between responders and non-responders in terms of glycosylation change. The dashed lines in FIG. 13 indicate a 50% glycosylation change between responders and non-responders. In summary, a majority of glycopeptides appeared closer to the zero-solid line, indicating that the increase in glycosylation upon epalrestat treatment between responders and non-responders was similar for a majority of the glycopeptides. Names and plausible structures drawn with glycosylation site are provided for some glycopeptides that showed a differential increase in glycosylation.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-8. (canceled)

9. A method for treating a mammal identified as (a) having a CDG and (b) having a biological sample containing an elevated level of one or more polyols, relative to the level of said one or more polyols in a biological sample from a control mammal not having said CDG,
wherein said method comprises administering to said mammal a dose of an aldose reductase inhibitor (ARI) that is increased as compared to a dose of said ARI that would be administered to a mammal identified as having said CDG and as having a biological sample that does not contain an elevated level of said one or more polyols, relative to the level of said one or more polyols in said biological sample from said control mammal.

10. The method of claim 9, wherein said mammal to which said increased dose of ARI is administered is a human.

11. The method of claim 9, wherein said CDG is a Type II CDG.

12. The method of claim 9, wherein said CDG is PMM2-CDG.

13. The method of claim 9, wherein said one or more polyols comprise one or more of mannitol, sorbitol, maltitol, xylitol, and erythritol.

14. (canceled)

15. The method of claim 9, wherein said biological sample is a urine sample or a blood sample.

16. The method of claim 9, wherein said ARI comprises epalrestat, AT007, alrestatin, benurestat, epalrestat, fidarestat, imirestat, lidorestat, minalrestat, ponalrestat, ranirestat, risarestat, sorbinil, tolrestat, zenarestat, or zopolrestat.

17. The method of claim 9, wherein the elevated level of said one or more polyols is increased by at least 10% as compared to the level of said one or more polyols in the biological sample from said control mammal.

18. The method of claim 9, wherein said increased dose is from about 0.5 mg/kg/day to about 5 mg/kg/day.

19. The method of claim 9, wherein said increased dose is at least about 50% greater than a dose of the ARI previously administered to said mammal.

20. A method for treating a mammal identified as having a CDG, the method comprising:
(a) measuring a level of one or more polyols in a biological sample from said mammal, and determining that a measured level of at least one of said one or more polyols is elevated relative to a level of the one or more polyols in a biological sample from a control mammal that does not have said CDG, and
(b) administering to said mammal a dose of an ARI that is increased as compared to a dose of said ARI that would be administered to a mammal identified as having said CDG and as having a biological sample that does not contain an elevated level of said one or more polyols, relative to the level of said one or more polyols in said biological sample from said control mammal.

21. The method of claim 20, wherein said mammal to which said increased dose of ARI is administered is a human.

22. The method of claim 20, wherein said CDG is a Type II CDG.

23. The method of claim 20, wherein said CDG is PMM2-CDG.

24. The method of claim 20, wherein said one or more polyols comprise one or more of mannitol, sorbitol, maltitol, xylitol, and erythritol.

25. (canceled)

26. The method of claim 20, wherein said biological sample is a urine sample or a blood sample.

27. The method of claim 20, wherein said ARI comprises epalrestat AT007, alrestatin, benurestat, epalrestat, fidarestat, imirestat, lidorestat, minalrestat, ponalrestat, ranirestat, risarestat, sorbinil, tolrestat, zenarestat, or zopolrestat.

28. The method of claim 20, wherein the elevated level of said one or more polyols is increased by at least 10% as compared to the level of said one or more polyols in the biological sample from said control mammal.

29. The method of claim 20, wherein said increased dose is from about 0.5 mg/kg/day to about 5 mg/kg/day.

30. The method of claim 20, wherein said increased dose is at least about 50% greater than a dose of the ARI previously administered to said mammal.
